(19) United States
(12) Patent Application Publication
(10) Pub. No.: US 2007/0219128 A1
(43) Pub. Date: Sep. 20, 2007

(54) Medical and health-care uses of pufferfish type I collagen extract and processes for producing said extract

(75) Inventors: Dingding Chen, Nanjing (CN); Lizhong Gao, Nanjing (CN)

Correspondence Address: SEED INTELLECTUAL PROPERTY LAW GROUP PLLC, 701 FIFTH AVE, SUITE 5400, SEATTLE, WA, 98104, US

(73) Assignee: NANJING BESSON PHARMACY CO., LTD, Nanjing (CN)

(21) Appl. No.: 10/570,058

(22) PCT Filed: Aug. 27, 2004
(86) PCT No.: PCT/CN04/00996

§ 371 Date: May 29, 2007

(30) Foreign Application Priority Data
Aug. 28, 2003 (CN) 03152848.1

(51) Int. Cl. A61K 38/39; C07K 14/78

(52) U.S. Cl. 514/12; 530/356

(57) ABSTRACT

The present invention relates to the use of pufferfish type I collagen extract as effective ingredient in the manufacture of medicaments and health-care foods for prevention and treatment of the following diseases, wherein the main chemical components and active components of the pufferfish type I collagen extract are natural pufferfish type I collagen or denatured pufferfish type I collagen extract and partial hydrolysates thereof. The present invention further relates to processes for the production of said pufferfish type I collagen extract, immunological assay methods of said extract, and uses of said extract as effective ingredient in treatment and health-care.

### TECHNICAL FIELD

The present invention relates to the use of pufferfish type I collagen extract as effective ingredient in the manufacture of medicaments and health-care foods for prevention and treatment of the following diseases, wherein the main chemical components and active components of the pufferfish type I collagen extract are natural pufferfish type I collagen or denatured pufferfish type I collagen extract and partial hydrolysates thereof. The present invention further relates to processes for the production of said pufferfish type I collagen extract, immunological assay methods of said extract, and uses of said extract as effective ingredient in treatment and health-care.

### BACKGROUND OF THE INVENTION

According to the taxology of fishes, the “pufferfish” used in the present invention belongs to Osteichthyes, Tetrodontiformes, Tetrodontidae. Pufferfish is also called Swellfish, Balloonfish, fugu or Blowfish. Up to now, due to the historical and consuetudinary reasons in China, pufferfish is often miscalled as river dolphin, an aquatic mammalian, which belongs to Cetacean Platanistidae.

About the Sources of Tetrodotoxin and the Toxicity of Pufferfish:

Pufferfish is venomous, but tetrodotoxin is not produced by the pufferfish per se, i.e., the Pufferfish does not synthesize tetrodotoxin. Pufferfishes are migrating fish, they migrate from Oceans to inland rivers during the ovulation period of from April to June per year and return to Oceans after ovulation, and adolescent pufferfishes migrate to oceans in the same year. In fact, tetrodotoxin is produced by several marine microorganisms that parasitize in the body of Pufferfish and do not exist in fresh water environment of rivers and lakes. After these marine Mnicroorganisms parasitize in pufferfish, they synthesize and secrete tetrodotoxin that is then accumulated and stored in pufferfish body. Other experiments confirm that tetrodotoxin is from tetrodotoxin-producing marine life that is eaten by pufferfish. Several laboratories in england and Japan had done many researches for this aspect, and found that pufferfish bred and fed in artificial seawater or natural seawater without toxin-bearing marine life was free of tetrodotoxin. Further researches confirmed that the genome and cells of pufferfish are lack of gene family and synthases necessary for biosynthesis of tetrodotoxin.

About Physiology, Biochemistry and Pharmacology of Collagens:

At present, over 20 collagens are disclosed, and they all have triple stranded helix Structure or partial triple stranded helices structure. It is generally believed that they are main component of extracellular matrix and bring about effects of support, connection, protection and constitution. They are proteins with the highest content in human body and are distributed in almost all organs and tissues. However, the number of collagens is great and they always have a great molecular weight and a complex structure, so that the physiology, biochemistry, biological function and pathology of collagens are not fully understood so far.

Collagen:

Collagen is protein in chemical nature, and collagen molecules are constituted of collagen subunits (such as α- and β-peptide chains) consisting of over ten kinds of amino acids that are arranged in a certain order. The collagen molecule consists of three helical peptide chains that are interwound. Each of said peptide chain consists of about 1000 amino acid residues. Peptide chains are linked between each other by hydrogen bonds and a small number of covalent bonds to increase the stability. Collagen is insoluble in water, which is determined by this compact, hard triple stranded helix structure. When collagen is denatured by heating or is hydrolyzed, this triple stranded helix structure is loosen to form gelatin with a random and irregular coil structure and a greatly improved water solubility.

Gelatin:

The products produced by inreversibly denaturing and partially degrading and cracking collagen are called gelatin. Namely, gelatin is the denaturation and partial degradation product of collagen, and is composed of disordered collagen subunits without its original spatial structure and collagen peptides that are partially hydrolyzed products thereof. However, collagen product and gelatin product are identical in their main chemical components, i.e., they are collagenous proteins.

Type I Collagen

Many documents confirm that the major of proteins of skin and bone are collagens. The skin mainly contains type I collagen and little type III collagen, and they are about 90% based on all proteins of skin. Type III collagen mainly exists in embryonal skin, and its content in skin gradually decreases after birth and finally reaches a very low level. The increase of type III collagen in skin mainly occurs in topical scar tissues. Bone mainly contains type I collagen, while cartilage mainly contains type II collagen, and they are more than 90% based on the total bone proteins. Since skin and bone are easily obtainable from abundant sources, they are the best raw materials for the extraction and production of type I collagen, and this is well known and widely applied by the person skilled in the art. This is also the main theoretical basis of the present invention (: Miller, E. J. et al., in: Methods in Enzymol., vol. 82, Academic Press; Ven Der Rest, M., et al. Collagen family of proteins. FASEB J., 1991, 5:2814-2823). In Japan, Nagai, T., et al., (Collagen of the skin of ocellate pufferfish—Takifugu rubripes. Food Chemistry, 2002, 78:173-177) also pointed out that the major collagen of the skin of Fugu ocellatus is type I collagen and has a triplex subunit of [α1(I)]₂α2(I).

It is well known by the public in China that the oral administration of animal gelatins (donkey-hide glue, glue of tortoise plastron, glue of colla piscis) can regulate and enhance immune function of body, and enhance resistance of body. However, the pharmacological action mechanism is still unknown. These animal gelatins are denatured collagens and partially hydrolyzed products thereof in their chemical nature.

Another important preparation of collagen in medicine and health care is cartilage collagen from shark (type II collagen). WO 95/32722 and WO 96/23512, etc. disclose Processes for the production of shark cartilage collagen extracts and their medical uses, and relate to the anti-matrix metalloproteinase, anti-angiogenesis and anti-tumor activities of shark cartilage collagen extracts.

Thus, collagens have some unknown important functions and effects, besides their effects of support, connection, protection and constitution.

About the Production Process and use of Pufferfish Glue and Gelatin:

Industrial processes for the production of gelatins including pufferfish glue (i.e., Pufferfish skin gelatin) mainly include acid method, base method and enzyme method, and their production procedures generally comprise the following main steps: (1) pre-treating the raw material, comprising: (a) pre-treatment, comprising: previewing, classifying, rinsing, cutting and degreasing; (b) removing impurities, softening and expanding, which mainly include three methods: acid, base (impregnating in lime cream) and enzyme processes; (2) extraction of gelatin, comprising boiling glue, i.e., extracting gelatin with boiling water from the raw material for producing gelatin; and (3) treatment of glue liquid, comprising: filtrating, concentrating, antisepticising, drying and molding to finally obtain gelatin in form of sheet, powder or granule .

Many references discloses laboratory methods for the production of type I collagen by Separation and purification, but these methods usually employ a long and complex process, have a relatively low yield, are carried out under rigorous conditions (10° C. or below), contain residual reagents, possess unclear pharmacological and biological activity and unwarranted safety, and are not suitable for large scale industrial production (Colowick, S. P., Kaplan, N. O., Methods in Enzyrnol., vol. 82 and vol. 144, Academic Press Inc.). The document of the aforementioned Nagai, T. et al. in Japan discloses a process for the extraction of the type I collagen of the skin of Fugu ocellatus, comprising firstly treating pufferfish skin with 0.1 mol/L NaOH at 4° C. to remove non-collagen proteins and pigments, lyophilizing, degreasing by using 10% n-butanol for 2 days, and lyophilizing again, then extracting by using 0.5 mol/L acetic acid for 3 days, centrifuging the extract at 20,000×g for 1 hour, salting out the centrifugation supernatant by using 0.7 mol/L NaCl, continuously adding NaCl under condition of pH7.5 until the final salting-out concentration of 2.5 mol/L is reached, centrifuging, dialyzing to remove salt, lyophilizing to obtain an acid-soluble collagen (ASC), wherein the yield of ASC is 10.7% relative to the dry weight of raw material; centrifuging, precipitate redispersing in 0.5 mol/L acetic and digesting with a large amount of pepsin (the amount of enzyme is 10% by the ratio of weight to volume, w/v) for 48 hours, centrifuging, dialyzing the centrifugation supernatant for 3 days, centrifuging again, redissolving in 0.5 mol/L acetic acid, then salting out by using 0.7 mol/L NaCl, centrifuging, adding NaCl under condition of pH7.5 until the final salting-out concentration of 2.2 mol/L is reached, centrifuging, precipitate redissolving in 0.5 mol/L acetic acid, dialyzing to remove salt, lyophilizing to obtain a pepsin soluble collagen (PSC), wherein the yield of PSC is 44.7% relative to the dry weight of raw material; and purifying the product by using ion exchange column CM-Toyopearl 650M, wherein the results of SDS-polyacrylamide gel electrophoresis indicate that both ASC and PSC are type I collagen of Fugu ocellatus, and all the above extraction steps are carried out at 4° C. It can be seen that this process is very complex, contains many steps and devices, and is very time-consuming, and it needs more than 20 days to carry out the whole process.

Pufferfish skin (bone) is toxic and usually unedible (natural pufferfish skin and bone contain a middle level of tetrodotoxin), and is generally used for making medical and industrial gelatin and for leatherworking, such as hemostatic sponge, absorbable suture line, cosmetics, etc. However, their medical and health-care uses as mentioned in the present invention are never disclosed.

About Pathology, Physiology and Pharmacology of the Relevant Diseases:

Gastric ulcer has an incidence rate of 8-10% in population, and is a common and frequently occurring chronic disease. Although the pathological mechanism of digestive gastric ulcer is not fully understood, it is known at present the following three main etiological factors: (1) excessive hydrochloric acid secreted by gastric parietal cells; (2) impaired or insufficient gastric mucosal defense; and (3) infection of helicobacter pylori. Correspondingly, there are three main drugs for treatment of digestive gastric ulcer in clinic, but these drugs have only a single effect and may cause many adverse effects. Thus, it is a very important project and object for many pharmaceutical companies to develop drugs for treatment of gastric ulcer with high performance, quick effect, lasting action and less adverse effects.

The primary drug abuse in the world is alcohol abuse (insobriety and excessive drinking), which results in very serious consequences and can hardly be controlled. The alcohol abuse is the main etiological factor to cause alcoholic gastric ulcer, gastric hemorrhage, alcoholic hepatic fibrosis (which may develop to cause hepatic cirrhosis), etc. Ethanol and many drugs and chemicals may induce hepatic fibrosis or hepatic cirrhosis, and hepatic cirrhosis may further develop to cause liver cancer. Thus, it is necessary to develop drugs with high performance, quick effect and low toxicity for prevention and treatment of alcoholic diseases (before and after drinking).

Present researches indicate that rheumatoid arthritis, rheumatic arthritis and lupus erythematosus are caused by disturbance and disorder of immune function in body, and are associated with the metabolism of (type II ) collagen.

One of the most important problems during the clinical chemotherapy is the side effects after chemotherapy (such as severe gastrointestinal reaction, body weight loss, immunity loss, leukocytopenia, etc.), which usually cause great physiological and psychological damages for patients, and such damages even are greater than the damages caused by the tumor per se. Thus, the use of drugs and health-care products that can increase the number of leukocytes, enhance the immunity of body and improve gastrointestinal function is an effective way to overcome the side effects of chemotherapy and to increase the effects of chemotherapy.

The present invention is generated exactly based on these basis and requirements.

### SUMMARY OF THE INVENTION

The object of the present invention is to provide a pufferfish type I collagen extract as effective ingredient in the manufacture of medicaments and health-care foods for prevention and treatment of the following diseases, wherein the main chemical components and active components of the pufferfish type I collagen extract are natural pufferfish type I collagen or denatured pufferfish type I collagen extract and partial hydrolysates thereof. The present invention further relates to processes for the production of said pufferfish type I collagen extract, immunological assay methods of said extract, and uses of said extract as effective ingredient in treatment and health-care.

Pharmacological Effects of the Pufferfish Type I Collagen Extract of the Present Invention:

We conducted many animal tests to study the pharmacodynamic and pharmacology of the pufferfish type I collagen extract of the present invention, and found that the pufferfish type I collagen extract has many pharmacological effects and biological activities. The results of some tests are as follows, and the following important conclusions of the pharmacological effects and pharmacodynamic of the pufferfish type I collagen extract of the present invention are obtained.
(1) The pufferfish type I collagen extract dose-dependently exhibits very significant protection effects on rat gastric ulcer and gastric mucosa damage induced by anhydrous ethanol.
(2) The pufferfish type I collagen extract dose-dependently exhibits very significant prevention effects on Shay rat gastric ulcer, which indicates that it has significant prevention and treatment effects on digestive gastric ulcer.
(3) The pufferfish type I collagen extract dose-dependently exhibits very significant treatment effects on rat gastric ulcer induced by acetic acid burn, which indicates that pufferfish type I collagen extract has treatment effects on chronic gastric ulcer and can significantly promote the healing of ulcer sites.
(4) The pufferfish type I collagen extract dose-dependently exhibits very significant protection effects on mouse gastric ulcer induced by reserpine, which indicates it has significant prevention and treatment effects on gastric ulcer caused by splenic asthenia.
(5) The pufferfish type I collagen extract dose-dependently exhibits very significant protection effects on rat gastric ulcer induced by indomethacin, which indicates it has significant prevention and treatment effects on gastric ulcer and gastric mucosa damage induced by non-steroid anti-inflammatory drugs.
(6) The pufferfish type I collagen extract dose-dependently exhibits very significant reduction effects on plasma transaminase activity elevation caused by acute rat liver injury induced by tetrachloromethane and anhydrous ethanol.
(7) The pufferfish type I collagen extract exhibits significant treatment effects on rat colonitis induced by 2,4,6-trinitrobenzene sulfonic acid (TNBS) and acetic acid, and on diarrhea caused thereby, and can be used for treatment of body weight loss caused by colonitis.
(8) The pufferfish type I collagen extract dose-dependently increases the number of leukocytes and platelets of mouse that is reduced by cyclophosphamide, which indicates it can enhance the immunity of body and reduce the side effects of chemotherapy.
(9) The pufferfish type I collagen extract dose-dependently inhibits the alcoholic fatty liver of rat and the pathologic increase of gastric wall collagen content, which indicates pufferfish type I collagen extract can inhibit the pathologic synthesis of collagen in liver and gaster, and then can prevent and treat hepatic fibrosis.
(10) Pufferfish type I collagen extract reaches 96.81% of the maximum drug action after 30 minutes of the administration, which means that the pufferfish type I collagen extract brings about quick effect; and it maintains 77.78% of the maximum drug action after 18 hours of the administration, which means that pufferfish type I collagen extract has a lasting action.
(11) The pufferfish type I collagen extract dose-dependently exhibits very significant inhibition effects on the mouse gastric emptying and the gastric emptying promoted by pyridostigmine bromide, which indicates the pufferfish type I collagen extract can block the action of acetylcholine, inhibit the contractile stimulation of acetylcholine, inhibit gastrospasm, prolong the retention time of food in gastrointestinal tract, and promote the digestion and absorption of food.
(12) The pufferfish type I collagen extract can significantly increase the levels of gastric mucosa prostaglandin E₂ (PGE₂) and prostacyclin-6-K that are reduced by indomethacin, which indicates that the pufferfish type I collagen extract can protect and maintain gastric mucosa PGE₂ and gastric mucosa blood flow which is one of the most important mechanisms for treatment of ulcer and for protection of gastric mucosa cells. The pufferfish type I collagen extract can further significantly improve the secretion of gastric mucosa (mucus).
(13) The pufferfish type I collagen extract dose-dependently exhibits very significant inhibition effects on rat gastric acid secretion stimulated by histamine and acetylcholine, and significantly inhibits the basic gastric acid secretion, which indicates that the blocking effect of the pufferfish type I collagen extract on histamine and acetylcholine is one of the important mechanisms for treatment of digestive gastric ulcer.
(14) The toxicological tests of Beagle dogs indicate that the pufferfish type I collagen extract is highly safe, and can be orally administrated for a long period with safety. The administration of a large dose of pufferfish type I collagen extract can increase the body weight, increase the weight of immune organs, and enhance the immunity of body.
(15) The pufferfish type I collagen extract dose-dependently exhibits very significant prevention and treatment effects on rat duodenal ulcer induced by cysteamine, acetic acid or histamine chloride/indomethacin respectively.
(16) The pufferfish type I collagen extract dose-dependently exhibits very significant inhibition effects on the increase of gastrin level in Shay rat blood serum, which indicates that the pufferfish type I collagen extract can inhibit the gastric acid secretion stimulated by gastrin, which is one of the important mechanisms for treatment of digestive gastric ulcer.
(17) On the one hand, the pufferfish type I collagen extract dose-independently reduces very significantly the nitric oxide (NO) level, iNOS activity and iNOS gene expression in rat gastric mucosa injured by ethanol and reduces very significantly the NO content and iNOS activity to a level below the normal level. On the other hand, the pufferfish type I collagen extract very significantly increases the cNOS gene expression that is reduced by the induction of ethanol, and recovers it to the normal level. These indicate that pufferfish type I collagen extract can discriminately regulate the NO level, iNOS activity, iNOS and cNOS gene expression in gastric mucosa, which is one of the important mechanisms for the protection of gastric mucosa, the vasodilatation, the improvement of blood flow of gastric mucosa, and the prevention and treatment of gastric ulcer.
(18) The pufferfish type I collagen extract can significantly inhibit the neovascularization of chick embryo.
(19) The pufferfish type I collagen extract significantly reduce in vitro the prothrombin time (PT), plasma thrombin time (TT), plasma activating partial thromboplastin time (APTT).
(20) The pufferfish type I collagen extract has a certain inhibition effects on H⁺, K⁺-ATPase of pig and rabbit in vitro.

Based on the above discoveries, the following conclusions are obtained in the present invention: the pufferfish type I collagen extract as an effective ingredient can be used for the prevention and treatment of the following diseases: gastrointestinal diseases, such as gastric ulcer, alcohol- and drug-induced gastric ulcer and gastrorrhagia, alcohol- and drug-induced gastric mucosa injury, stress gastric ulcer, acute and chronic gastritis, superficial and erosive gastritis, gastrospasm, gastralgia, bile reflux gastric ulcer, duodenal ulcer, irritable bowel syndrome, colonitis, gastrointestinal dysfunction, gastric kinetics disorder, indigestion and malabsorption, and body weight loss, abdominal distension and diarrhea caused thereby; liver cell damage and collagen proliferative diseases, such as alcoholic liver damage and hepatic fibrosis and hepatic cirrhosis caused thereby, hepatic fibrosis, hepatic cirrhosis, drug-induced liver damage and hepatic fibrosis and hepatic cirrhosis caused thereby, kidney fibrosis, myocardial fibrosis; immune diseases, such as immune dysfunction and decrease, leucopenia, rheumatoid arthritis, rheumatic arthritis, lupus erythematosus; tumors, such as the occurrence, development and metastasis of malignant gastric tumors, gastric cancer, liver cancer, colon cancer, rectal cancer, and the occurrence, development and metastasis of other solid malignant tumors, and angiogenesis-associated diseases.

So far, there is no report in China and other countries that relates to the animal tests and clinical application of pufferfish glue, pufferfish type I collagen that have the biological activity, pharmacological effects and mechanisms in medical and health-care uses as mentioned in the present invention. The inventors of the present invention firstly disclose the pufferfish type I collagen extract has these pharmacological activities and complete the present invention.

The present invention further provides a process for the production of pufferfish type I collagen extract by using pufferfish skin and/or pufferfish bone including fins, said process comprising:

(1) Pre-Treating Raw Materials:

(a) pre-treating natural pufferfish skin and bone raw material to remove toxin: treating the raw material in an acid solution or alkaline solution at 0 to 50° C. for 4 to 48 hours, sufficiently washing with water, and repeating this step for 4 to 6 times;
wherein when an alkaline solution is used for the removal of toxin, the preferred conditions are: normal pressure, a final alkaline solution concentration of 0.01 to 0.1 mol/L, 20-30° C., detoxifying for 6 to 24 hours, and repeating for 4 to 5 times; and when an acid solution is used for the removal of toxin, the preferred conditions are: normal pressure, a final acid solution concentration of 0.1 to 0.2 mol/L, 0-20° C., detoxifying for 6 to 24 hours, and repeating for 4 to 5 times;
(b) washing clearly the skin and bone of the pufferfish artificially bred in fresh water or the detoxified skin and bone of natural pufferfish with water, and storing at −20° C. or below for standby if it is not used immediately;
   (2) Extracting According to one of the Following Three Methods:
(a) adding water or acid solution to the pre-treated raw material of pufferfish skin and bone in any proportion, extracting at a temperature of from 0 to 125° C., a pressure of from normal pressure to 3 atm for 60 minutes to 100 hours, filtering to obtain the liquid portion, repeating for 0 to 6 times, combining the filtrates, adding water to the residue or combining the filtrates with the residue, homogenizing to obtain a homogenate, standing the homogenate obtained by the acid solution as extracting solvent at 20° C. or below for 12 to 48 hours; The homogenate obtained by water as extracting solvent will be directly used in the next step;
wherein when the acid solution is used for the extraction, the preferred conditions are: normal pressure, 0 to 10° C., a final acid solution reaction concentration of 0.1 to 0.5 mol/L, extracting for 48 to 72 hours, repeating for 2 to 4 times, homogenizing; and normal pressure, 40 to 80° C., a final acid solution reaction concentration of 0.01 to 0.2 mol/L, extracting for 4 to 8 hours, repeating for 3 to 5 times, and homogenizing;
wherein when water is used for the extraction, the preferred conditions are: normal pressure, 90 to 100° C., extracting for 3 to 8 hours, repeating for 1 to 3 times, and homogenizing;
(b) adding water or acid solution to the pre-treated pufferfish bone raw material, extracting at a temperature of from 0 to 125° C. and a pressure of from normal pressure to 3 atms for 60 minutes to 100 hours, filtering to obtain the liquid portion, repeating for 0 to 6 times, combining filtrates, discarding residue, concentrating the filtrates to 100% to 10% of the original volume, adding an amount of pufferfish skin raw material, extracting at a temperature of from 0 to 125° C. and a pressure of from normal pressure to 3 atms for 60 minutes to 100 hours, filtering to obtain the liquid portion, adding water or the same acid solution and repeating for 0 to 6 times, combining filtrates, combining the filtrates and the residue, homogenizing to obtain a homogenate, standing the homogenate obtained by the acid solution as extracting solvent at 20° C. or below for 12 to 48 hours; The homogenate obtained by water as extracting solvent will be directly used in the next step;
wherein when the acid solution is used for the extraction, the preferred conditions are: normal pressure, 0 to 10° C., a final acid solution reaction concentration of 0.1 to 0.5 mol/L, extracting for 48 to 72 hours, repeating for 2 to 4 times, homogenizing; and normal pressure, 40 to 80° C., a final acid solution reaction concentration of 0.01 to 0.2 mol/L, extracting for 4 to 8 hours, repeating for 3 to 5 times, and homogenizing;
wherein when water is used for the extraction, the preferred conditions are: normal pressure, 90 to 100° C., extracting for 3 to 8 hours, repeating for I to 3 times, and homogenizing;
(c) obtaining the pufferfish type I collagen extract of the present invention by the conventional methods or modified methods for extracting type I collagen and gelatin in the prior art; for example, treating the pretreated raw material of pufferfish skin and bone in any proportion with dilute base, dilute acid or proteinase for over 24 hours to remove other protein impurities, washing with water, degreasing, repetitively extracting at 10° C. or below with a dilute acid such as 0.1-0.5 mol/L acetic or hydrochloric acid, homogenizing, centrifuging, neutralizing or not neutralizing the centrifugation supernatant and dilute acid extract, salting-out stepwise and repetitively by using 0.7-4.4 mol/L neutral salt such as sodium chloride to obtain a precipitate of type I collagen; or repetitively extracting at 10° C. or below by using a dilute solution of neutral salt such as sodium chloride, centrifuging or filtering to obtain an extract of type I collagen; hydrolyzing the extracted residue with a protease such as pepsin, performing again the above salting-out step to obtain pufferfish type I collagen; purifying the type I collagen extract solution by a DEAE- or CM-ion exchanger to remove other protein impurities to obtain pufferfish type I collagen (however, when the process for the extraction of pufferfish type I collagen in the prior art (see the document of Nagai, T.) is employed to extract the pufferfish type I collagen extract of the present invention, the production period is long, the energy consumption is large, said process is unsuitable for the large scale production in industry, the pharmacological activity of the product is lower and depends on the conditions, and said process generates the contaminative wastes);
   (3) Filtrating and Concentrating:
centrifuging or filtering the homogenate to remove residue, optionally concentrating the filtrate to 100% to 10% of the original volume to obtain a concentrated pufferfish type I collagen extract;
wherein when the extraction is carried out by using acid solution at low temperature, the preferred method for removing residue is high speed centrifugation at a low temperature; while when the extraction is carried out by using water or the acid solution at high temperature, the preferred method for removing residue is filtration;
when the extraction is carried out by using acid solution at low temperature, the preferred concentration method is the concentration by ultrafiltration using a ultrafiltration membrane with a pore diameter of 100 to 200 Kda; when the extraction is carried out by using water or acid solution at high temperature, the preferred concentration method is vacuum concentration;
the preferred simple production method comprises: after the aforementioned extract is centrifuged or filtrated to remover residue, it is directly subjected to (ultrafiltration) concentration and (freeze, spray) drying to obtain the pufferfish type I collagen extract;
   (4) Optionally, Drying and Pulverizing:
drying the extract or the concentrated extract (spray-drying, freeze-drying, or microwave-drying, drying by baking, drying in the shade, preferably freeze-drying or spray-drying), pulverizing to obtain the pufferfish type I collagen extract, a light-yellow or white powdery product capable of passing through a 80 mesh sieve;
wherein the acid solution is an organic acid or inorganic acid; the alkaline solution is an inorganic base; the final concentration during the extraction is 0.001 to 1.0 mol/L; the final concentration during the detoxification is 0.01 to 0.5 mol/L; the examples of the used acid are: formic acid, acetic acid, propionic acid, malonic acid, butyric acid, succinic acid, malic acid, citric acid, tartaric acid, lactic acid, phosphoric acid, hydrochloric acid, sulfuric acid, nitric acid; the examples of the used alkali are: sodium hydroxide, potassium hydroxide, calcium hydroxide (lime water), sodium carbonate; the examples of the used enzyme are: trypsin, pancreatin, pepsin, papain, chymotrypsin, bromelain, dispase, pronase, fibrin, gelatinase, type II collagenase, type III collagenase, proteinase K, and various proteinases from other animals, plants and microorganisms.

In a preferred embodiment, the controllable partial hydrolysis is performed according to the following two methods after the steps of filtration and concentration:
(1) hydrolyzing by using a proteinase under conditions of: a proteinase concentration of 1-100 mg/100 g wet weight tissue, preferably 10-50 mg/100 g wet weight tissue in the reaction system, stirring, a temperature of 20-65° C., preferably 30-37° C., a time of 3-100 hours, preferably 3-48 hours, heating to 100° C. for 5-10 minutes to terminate the enzyme activity after the end of enzymolysis;
(2) hydrolyzing by using an organic acid and/or an inorganic acid under conditions of: an acid concentration of 0.001-1.0 mol/L , preferably 0.05-0.50 mol/L in the reaction system, stirring, a temperature of 0-100° C., preferably 25-75° C., a time of 60 minutes to 72 hours, preferably 3 to 24 hours, neutralizing or removing acid under vacuum;
wherein the examples of the used acid are: formic acid, acetic acid, propionic acid, malonic acid, butyric acid, succinic acid, malic acid, citric acid, tartaric acid, lactic acid, phosphoric acid, hydrochloric acid, sulfuric acid, nitric acid; the examples of the used alkali are: sodium hydroxide, potassium hydroxide, calcium hydroxide (lime water), sodium carbonate; the examples of the used enzymes are: trypsin, pancreatin, pepsin, papain, chymotrypsin, bromelain, dispase, pronase, fibrin, gelatinase, type II collagenase, type III collagenase, proteinase K, and various proteinases from other animals, plants and microorganisms; the preferred enzyme is type III collagenase, trypsin, pepsin; the preferred acid is acetic acid, hydrochloric acid.

Optionally, the hydrolyte is concentrated to a volume of 100% to 10% of the original volume, and drying the concentrated hydrolyte or precipitating and drying to obtain the pufferfish type I collagen extract.

In a further preferred embodiment, the precipitation is performed according to the following two methods after the step of concentration:
(1) adding to the concentrated extract cold acetone having a volume of 8 to 15 times, preferably 10 to 12 times the volume of the concentrated extract, sedimentating at 10° C. or below for 24 to 48 hours, centrifuging or filtering to obtain a precipitate, volatilizing the residual organic solvent from the precipitate, and drying to obtain the pufferfish type I collagen extract;
(2) adding to the concentrated extract cold ethanol until the final ethanol concentration reaches 55-90%, preferably 75-90%, sedimentating at 10° C. or below for 24-48 hours, centrifuging or filtering to obtain a precipitate, volatilizing the residual organic solvent from the precipitate, optionally drying to obtain the pufferfish type I collagen extract; preferably, repetitively extracting the obtained precipitate with a neutral buffer (pH7.5) containing 1.0-2.2 mol/L NaCl or directly with a 1.0-2.2 mol/L NaCl solution, desalting the extract, optionally drying to obtain a pufferfish type I collagen extract with a relatively high purity;
more preferably, purifying the extract obtained in the above step by DEAE- and/or CM-ion exchange method to remove protein impuiteis, desalting the ion exchange elution liquid, and drying to obtain a pufferfish type I collagen extract with high purity.

The process for the production of the pufferfish type I collagen extract of the present invention is illustrated more detailedly as follows:
(1) Pre-Treating Raw Materials:
(a) pre-treating natural pufferfish skin and bone raw material to remove toxin: treating the raw material in an acid solution or alkaline solution at 0 to 50° C. for 4 to 48 hours, sufficiently washing with water, and repeating this step for 4 to 6 times;
wherein when the alkaline solution is used for the removal of toxin, the preferred conditions are: normal pressure, a final alkaline solution concentration of 0.01 to 0.1 mol/L, 20-30° C., detoxifying for 8 to 24 hours, and repeating for 4 to 5 times; and when an acid solution is used for the removal of toxin, the preferred conditions are: normal pressure, a final acid solution concentration of 0.1 to 0.2 mol/L, 0-20° C., detoxifying for 6 to 24 hours, and repeating for 4 to 5 times;
(b) washing clearly the skin and bone of the pufferfish artificially bred in fresh water or the detoxified skin and bone of natural pufferfish with water, and storing at −20° C. or below for a long period for standby if it is not used immediately;
(2) Extracting According to One of the Following Three Methods:
a) adding water or an acid solution to the pre-treated raw material of pufferfish skin and bone in any proportion, extracting at a temperature of from 0 to 125° C., a pressure of from normal pressure to 3 atms for 60 minutes to 100 hours, filtering to obtain the liquid portion, repeating for 0 to 6 times, combining the filtrates, adding water to the residue or combining the filtrates with the residue, homogenizing to obtain a homogenate, standing the homogenate obtained by the acid solution as extracting solvent at 20° C. or below for 12 to 48 hours; The homogenate obtained by water as extracting solvent will be directly used in the next step;
wherein when the acid solution is used for the extraction, the preferred conditions are: normal pressure, 0 to 10° C., a final acid solution reaction concentration of 0.1 to 0.5 mol/L, extracting for 48 to 72 hours, repeating for 2 to 4 times, homogenizing; and normal pressure, 40 to 80° C., a final acid solution reaction concentration of 0.01 to 0.2 mol/L, extracting for 4 to 8 hours, repeating for 3 to 5 time, and homogenizing; wherein when water is used for the extraction, the preferred conditions are: normal pressure, 90 to 100° C., extracting for 3 to 8 hours, repeating for 1 to 3 times, and homogenizing;
b) adding water or an acid solution to the pre-treated pufferfish bone raw material, extracting at a temperature of from 0 to 125° C. and a pressure of from normal pressure to 3 atms for 60 minutes to 100 hours, filtering to obtain the liquid portion, repeating for 0 to 6 times, combining filtrates, discarding residue, concentrating the filtrates to a volume of 100% to 10% of the original volume, adding an amount of pufferfish skin raw material, extracting at a temperature of from 0 to 125° C. and a pressure of from normal pressure to 3 atms for 60 minutes to 100 hours, filtering to obtain the liquid portion, adding water or the same acid solution and repeating for 0 to 6 times, combining filtrates, combining the filtrates and the residue, homogenizing to obtain a homogenate, standing the homogenate obtained by the acid solution as extracting solvent at 20° C. or below for 12 to 48 hours; The homogenate obtained by water as extracting solvent will be directly used in the next step; wherein when the acid solution is used for the extraction, the preferred conditions are: normal pressure, 0 to 10° C., a final acid solution reaction concentration of 0.1 to 0.5 mol/L, extracting for 48 to 72 hours, repeating for 2 to 4 times, homogenizing; and normal pressure, 40 to 80° C., a final acid solution reaction concentration of 0.01 to 0.2 mol/L, extracting for 4 to 8 hours, repeating for 3 to 5 times, and homogenizing; wherein when water is used for the extraction, the preferred conditions are: normal pressure, 90 to 100° C., extracting for 3 to 8 hours, repeating for 1 to 3 times, and homogenizing.

This step directly obtains a pufferfish type I collagen extract from a pre-treated mixture of pufferfish skin and bone (fins) in any proportion, or firstly obtains an extract from pufferfish bone (fins) and then obtains a pufferfish type I collagen extract from pufferfish skin by using the firstly obtained extract as extracting solvent. Said step is mainly characterized in that the type I collagen in raw material is directly extracted by using water or a dilute acid solution at a relatively high temperature, and the type I collagen is fully extracted for 12 to 48 hours after the homogenization; or the raw material is extracted by a dilute weak acid solution at a low temperature, and then the type I collagen is fully extracted for 12 to 48 hours, which ensure the short production period and high efficiency of the process of the present invention, This is one inventive step of the extraction process of the present invention. Said extraction process utilizes two important differences in solubility between the type I collagen and other proteins: (1) the type I collagen is soluble in dilute acid solution, in particular when the temperature of the dilute acid solution is greater than 40° C., the type I collagen has a very high solubility and is almost fully dissolved in the acid solution, while most other proteins have a decreased solubility at this temperature and form insoluble denatured protein precipitate so that these proteins are separated from the type I collagen, which facilitates their removal by centrifugation or filtration; and (2) the heat denatured type I collagen is soluble in hot water, while other heat denatured proteins form a precipitate and are completely separated from the type I collagen; on the other hand, it is found in the present invention that the pharmacological activity of pufferfish type I collagen extract is very stable after it is heated in a water solution at a temperature of below 100° C. or in a dilute acid solution at a temperature of below 80° C. for several hours; these are innovative and unique features of the process of the present invention for producing pufferfish type I collagen extract, and are theoretic basis of the present invention to produce pufferfish type I collagen extract with high performance; on the contrary, the process in the prior art merely conducts a long-time extraction by using a dilute acid at a low temperature, and uses a large amount of pepsin for hydrolysis and extraction (which simultaneously introduces exogenous other protein impurities), or employs a treatment by using a alkaline solution for a long time to remove other protein impurities and then using boiling water for repetitive extraction, so that the process in the prior art has disadvantages of long time, high energy consumption, high cost, generation of the three wastes, and affected product activity.
c) obtaining the pufferfish type I collagen extract of the present invention by the conventional methods or modified methods for extracting type I collagen and gelatin in the prior art [Nagai, T. et al. Collagen of the skin of ocellate pufferfish (Takifugu rubripes). Food Chemistry, 2002, 78:173-177]; for example, treating the pretreated raw material of pufferfish skin and bone in any proportion with dilute base, dilute acid or proteinase to remove other protein impurities, washing with water, degreasing, repetitively extracting at 10° C. or below with a dilute acid such as 0.1-0.5 mol/L acetic acid or hydrochloric acid, homogenizing, centrifuging, neutralizing or not neutralizing the centrifugation supernatant and the dilute acid extract, salting-out stepwise and repetitively by using 0.7-4.4 mol/L neutral salt such as sodium chloride to obtain a precipitate of type I collagen; or repetitively extracting at 10° C. or below by using a dilute solution of neutral salt such as sodium chloride, and filtering to obtain an extract of type I collagen; or repetitively extracting by using boiling water, and filtering to obtain an extract of the denatured type I collagen; hydrolyzing the extracted residue with a protease such as pepsin, performing again the above salting-out step to obtain pufferfish type I collagen; purifying the type I collagen extract solution by a DEAE- or CM-ion exchanger to remove other protein impurities to obtain pufferfish type I collagen (however, according to the above statement, when the process for the extraction of pufferfish type I collagen in the prior art is employed to extract the pufferfish type I collagen extract of the present invention, the production period is long, the energy consumption is large, the cost is high, and contaminative wastes are generated, and the pharmacological activity of the product depends on the conditions);
(3) Filtrating and Concentrating:
centrifuging or filtering the homogenate to remove residue, concentrating the filtrate to a volume of 100% to 10% of the original volume to obtain a concentrated pufferfish type I collagen extract;
wherein when the extraction is carried out by using acid solution at low temperature, the preferred method for removing residue is high speed centrifugation at a low temperature; while when the extraction is carried out by using water or the acid solution at high temperature, the preferred method for removing residue is filtration;
when the extraction is carried out by using acid solution at low temperature, the preferred concentration method is the concentration by ultrafiltration using a ultrafiltration membrane with a pore diameter of 100 to 200 Kda; when the extraction is carried out by using water or acid solution at high temperature, the preferred concentration method is vacuum concentration;
(4) Optionally, Drying and Pulverizing:
drying the concentrated extract by spray-drying, freeze-drying, or drying by baking, or drying in the shade, pulverizing to obtain the pufferfish type I collagen extract, a light-yellow or white powdery product capable of passing through a 80 mesh sieve, which mainly comprises pufferfish type I collagen or denatured pufferfish type I collagen and partially hydrolyzed pufferfish type I collagen; wherein the preferred drying method is spray-drying or freeze-drying;
wherein the acid solution is an organic acid or inorganic acid; the alkaline solution is an inorganic base; the final concentration during the extraction is 0.001 to 1.0 mol/L; the final concentration during the detoxification is 0.01 to 0.5 mol/L; the examples of the used acid are: formic acid, acetic acid, propionic acid, malonic acid, butyric acid, succinic acid, malic acid, citric acid, tartaric acid, lactic acid, phosphoric acid, hydrochloric acid, sulfuric acid, nitric acid; the examples of the used alkali are: sodium hydroxide, potassium hydroxide, calcium hydroxide (lime water), sodium carbonate; the examples of the used enzyme are: trypsin, pancreatin, pepsin, papain, chymotrypsin, bromelain, dispase, pronase, fibrin, gelatinase, type II collagenase, type III collagenase, proteinase K, and various proteinases from other animals, plants and microorganisms.

Since the extraction steps have selectivity for pufferfish type I collagen and the raw materials mainly comprises type I collagen, a pufferfish type I collagen extract is obtained by centrifuging or filtering the above extract to remove residue, and directly (ultrafiltration) concentrating, (freeze-, spray) drying. Thus, a simple and convenient process for production of pufferfish type I collagen extract is established. This is one of inventive points of the present invention and is not found in the process for the production of pufferfish glue in the prior art.

After the about extraction steps and concentration steps, the controllable partial hydrolysis is performed according to the following two methods:
(1) hydrolyzing by using a proteinase under conditions of: a proteinase concentration of 1-100 mg/100 mg wet weight tissue, preferably 10-50 mg/100 g wet weight tissue in the reaction system, stirring, a temperature of 20-65° C., preferably 30-37° C., a time of 3-100 hours, preferably 3-48 hours, heating to 100° C. for 5-10 minutes to terminate the enzyme activity after the end of enzymolysis;
(2) hydrolyzing by using an organic acid and/or an inorganic acid under conditions of: an acid concentration of 0.001-1.0 mol/L , preferably 0.05-0.50 mol/L in the reaction system, stirring, a temperature of 0-100° C., preferably 25-75° C., a time of 60 minutes to 72 hours, preferably 3 to 24 hours, neutralizing or removing acid under vacuum; wherein the preferred enzyme is type III collagenase, trypsin, pepsin; the preferred acid is acetic acid, hydrochloric acid. The process for production of pufferfish glue in the prior art does not include this hydrolyzing step, but the controllable hydrolysis of pufferfish type I collagen extract is very important for the production of a product with high pharmacological activity. This is a further feature of the process of the present invention.

The hydrolyte is concentrated to a volume of 100% to 10% of the original volume, and the pufferfish type I collagen extract is obtained by (spray, freeze) drying the concentrated hydrolyte, or by sedimentation according to one of the following two sedimentation methods. The precipitate is dried to obtain a pufferfish type I collagen extract mainly comprising pufferfish type I collagen or denatured pufferfish type I collagen and partial hydrolytes thereof.

After the above concentration step, the sedimentation is performed according to the following two methods:
(1) adding to the concentrated extract a cold acetone having a volume of 8 to 15 times, preferably 10 to 12 times the volume of the concentrated extract, sedimentating at 10° C. or below for 24 to 48 hours, centrifuging or filtering to obtain a precipitate, volatilizing the residual organic solvent from the precipitate, and drying to obtain the pufferfish type I collagen extract;
(2) adding to the concentrated extract a cold ethanol until the final ethanol concentration reaches 55-90%, preferably 75-90%, sedimentating at 10° C. or below for 24-48 hours, centrifuging or filtering to obtain a precipitate, volatilizing the residual organic solvent from the precipitate, optionally drying to obtain the pufferfish type I collagen extract. All processes in the prior art for the extraction of pufferfish type I collagen extract do not employ cold acetone or ethanol to treat precipitate, while such sedimentation method can be readily industrialized, has a high sedimentation efficiency, can recover and reuse acetone and ethanol, and can readily remove the residual reagents in the product. On the contrary, the sedimentation efficiency of NaCl salting-out is lower, and a large amount of NaCl and a part of other protein impurities precipitate together with the product, so that sequent steps for removing them are needed. This sedimentation step is a further feature of the process of the present invention.

In the present invention, the precipitate obtained from said sedimentation step can be repetitively extracted in a reverse-direction manner by a neutral buffer (pH7.5) containing 1.0-2.2 mol/L NaCl or directly by a 1.0-2.2 mol/L NaCl solution, desalting the extract, and dried to obtain a pufferfish type I collagen extract with a relatively high purity. The processes for the extraction and production of pufferfish glue or pufferfish type I collagen in the prior art do not include this step. This step is a further feature of the present invention.

Since pufferfish type I collagen extract is a protein product, it can be detected and quantified by specific immune methods with high sensitivities, such as immunodiffusion method, counter immunoelectrophoresis, immune nephelometric analysis, solid-phase enzyme-linked immunospot method (ELISPOT), ELISA and RIA. The present invention firstly discloses various immunoassay methods of pufferfish type I collagen extract.

The pufferfish type I collagen extract in the art of the present invention has the following features:
(1) The pufferfish type I collagen extract is prepared by using pufferfish skin and/or bone (fins) as raw materials, comprises pufferfish type I collagen or denatured type I collagen and partial hydrolytes thereof as main chemical components and pharmacologically active components, and have the pharmacological and biological activities as mentioned in claim 1, and typical physical/chemical properties of (fish) type I collagen (see FIGS. 1 to 4, Tables 1-3 and 5);
(2) The content of pufferfish type I collagen or denatured pufferfish type I collagen and partial hydrolytes thereof in the pufferfish type I collagen extract is greater than 50%, and the total protein content is greater than 70%;
(3) The molecular weight of pufferfish type I collagen trimer [α1(I)]2α2(I) is from 300 to 420 KDa, and the molecular weight of the denatured pufferfish type I collagen (comprising α1(I) monomer, α2(I) monomer, α1(I)2 dimer and α1(I)α2(I) dimer) and partial hydrolytes thereof is from 60 to 300 KDa; the isoelectric points of the two subunits of the pufferfish type I collagen protein separately are α1(I):4.85±0.5 and α2(I):6.71±0.5 (see FIG. 1), while the isoelectric points of the two subunits of pufferfish type I collagen may vary according to the species of pufferfish;
(4) The results of the ultraviolet absorption scanning show that the maximum wavelength of ultraviolet absorption of 0.3 mg/ml pufferfish type I collagen extract solution obtained by using 0.2 mol/L acetic acid as solvent is 226±3 nm; while the maximum wavelength of ultraviolet absorption of 0.1 mg/ml pufferfish type I collagen extract solution obtained by using 0.1 mol/L hydrochloric acid as solvent is 203±3 nm; and further, there is no absorption peak in the range from 260 to 280 nm and the absorption value in said wavelength range is relatively low (see FIG. 3);
(5) According to the measurements of Kivirikko method and automatic amino acid analyzer, the pufferfish type I collagen extract has a weight percentage content of hydroxyproline of greater than 4.5% similar to other fish collagen; the weight percentage content of hydroxyproline of fish collagen is usually lower than 10%, which is significantly lower than the content of hydroxyproline (14%) in the collagen of terrestrial animal; the amino acid components of the pufferfish type I collagen extract obtained by the process of the present invention are shown in Tables 1, 2, 3 and 5; the pufferfish type I collagen extract is glycoprotein and has a protein-bound carbohydrate content of 0.5 to 1.9%; and it is understandable that the data difference is caused by different raw materials and different extraction conditions, but they are measured based on pufferfish type I collagen as main chemical component and pharmacologically active component;
(6) The pufferfish type I collagen extract is soluble in water and dilute acid solution, wherein the water solution is thermostable and can maintain its pharmacological activity after being heated at 95 to 100° C. for several hours; the dilute weak acid solution of pufferfish type I collagen extract (less than 0.5 mol/L) maintains a substantially stable pharmaceutical activity after being placed at a temperature from −20° C. to room temperature for a long period; however, the pufferfish type I collagen extract is very sensitive for alkali and may fully lose its pharmaceutical activity in a weak alkali solution, even it is merely placed at room temperature for several hours, while the pufferfish type I collagen extract in the pufferfish skin and bone tissues is relatively stable at low temperature; the pufferfish type I collagen extract is not sensitive for type III collagenase, and is sensitive for type I collagenase; after the pufferfish type I collagen extract is hydrolyzed by a type I collagenase, its pharmacological activity decreases quickly.

The acid solution used in the process of the present invention for the production of Pufferfish type I collagen extract is an organic acid or inorganic acid; the alkaline solution is an inorganic alkali; and there are many acids, bases and proteinases that can be used in the present invention. The examples of the used acid are: formic acid, acetic acid, propionic acid, malonic acid, butyric acid, succinic acid, malic acid, citric acid, tartaric acid, lactic acid, phosphoric acid, hydrochloric acid, sulfuric acid, nitric acid; the examples of the used alkali are: sodium hydroxide, potassium hydroxide, calcium hydroxide (lime water), sodium carbonate; the examples of the used enzyme are: trypsin, pancreatin, pepsin, papain, chymotrypsin, bromelain, dispase, pronase, fibrin, gelatinase, type II collagenase, type III collagenase, proteinase K, and various proteinases from other animals, plants and microorganisms.

The pufferfish as mentioned in the present invention is selected from all pufferfishes belong to Fugu, Tetraodontidae, Tetraodontoidei, including fugus such as Fugu obscurus, Fugu rubripes, Fugu pseudommus, Fugu xanthopterus, Fugu flavidus, Fugu basilewskianus, Fugu reticularis, Fugu porphyreus, Fugu vermicularis, Fugu bimaculatus, Fugu pardalis, Fugu niphobles, Fugu albopumbeus, Fugu oblongus, Fugu ocellatus, Fugu orbimaculatus, Fugu coronoidus, etc.; Arothrons such as Arothron stellatus, Arothron hispidus, Arothron nigropucntatus, etc.; and the following pufferfishes, including Liosaccus cutaneus, Ostracion tuberculatus, Mola mola, Masturus lanceolatus, Triacanthus brevirostris, Triacanthus strigilfera, Alutera monoceros, Diodon novemaculatus, Diodon holacanthus, Diodon hystix, Chilomycterus affinis, Gastrophysus gloveri, Gastrophysus lunaris. These pufferfishes are from nature seas, rivers and lacks, or are pufferfishes (including young Fugu obscurus) artificially bred in fresh water.

The pufferfish type I collagen extract can be processed to form various oral preparations as medicaments or health-care foods via methods well known in the art. The examples of specific preparations comprises: tablets (including coated tablets, plain tablets, gastric suspension tablets, buccal tablets, effervescent tablets and chewable tablets), capsules (including soft capsules and microcapsules), granules, powder preparations for infusion, effervescent granules, powders (including freeze-dried powders), pills (dripping pills), controlled release tablets (including enteric coated tablets, sustained-release tablets), controlled release capsules (including enteric coated capsules, and sustained-release tablets), fruit flavored preparations, chewable blocks, oral solutions, syrups, oral disintegrating preparations, suspensions, spray preparations, solutions (including broth), gels, emulsions, slurries, drops, etc.

The pufferfish type I collagen extract prepared according to the method of the present invention has the following 10 beneficial effects or features:
(1) The main chemical components and pharmacologically active substances of the pufferfish type I collagen extract of the present invention are nature type I collagen of pufferfish skin and bone (fins) or denatured pufferfish type I collagen protein and partial hydrolytes thereof, and they have many unexpected biological activities, so that the pufferfish type I collagen extract is promising and have many values in medical treatments and health-care applications.
(2) In particular, the predominant features of the pufferfish type I collagen extract of the present invention lies in the very significant effects in prevention, treatment and health care of gastrointestinal ulcers and inflammations, such as alcoholic gastric ulcer, alcoholic gastric mucosa damage, alcoholic gastric hemorrhage, alcoholic poisoning, drug-induced gastric ulcer, drug-induced gastric mucosa damage, drug-induced gastric hemorrhage, duodenal ulcer, irritable bowel syndrome, colonitis, acute and chronic gastritis, superficial gastritis, erosive gastritis, gastrospam, gastralgia.
   The pufferfish type I collagen extract can very significantly inhibit the secretion of gastrin, gastric acid and pepsin of model animals, which indicate that the pufferfish type I collagen extract has prevention and treatment effects on digestive gastric ulcer and gastric hyperacidity, and is a highly effective agent against gastric acid secretion.
   The pufferfish type I collagen extract can very significantly increase the levels of gastric mucosa prostaglandin E₂ (PGE₂) and prostacyclin-6-K, very significantly inhibit the inducible-nitric oxide synthase (iNOS) activity and its gene expression, significantly promote cNOS activity and its gene expression, inhibit the pathologic increase of the NO level generated by iNOS in gastric mucosa, and elevate the beneficial NO generated by cNOS, which indicate that the pufferfish type I collagen extract can protect gastric mucosa, relax blood vessels, improve blood flow of gastric mucosa, treat ischemic gastric mucosa damage and necrosis, and inhibit inflammations and gastrointestinal tumors via PGs and NO/NOS pathways. Thus, the pufferfish type I collagen extract is a high effective gastric mucosa protective agent.
(3) In the meantime, it is specifically pointed out that another predominant feature of the pufferfish type I collagen extract of the present invention lies in that it can inhibit pathologic accumulation of collagen in liver and stomach tissues, and significantly inhibit the angiogenesis (CAM test), which indicate that the pufferfish type I collagen extract can inhibit the abnormal synthesis of collagen in tissues, and can be used for prevention and treatment of alcoholic liver damage, alcoholic fatty liver, alcoholic liver cirrhosis, alcoholic liver fibrosis, fibrosis of lung and kidney tissues, collagen proliferation-associated diseases, angiogenesis-associated diseases, and the generation, development and metastasis of solid tumors. urther, the pufferfish type I collagen extract can significantly reduce the increases of blood plasma transaminase level induced by ethanol and drugs, which indicates that the pufferfish type I collagen extract has effects against the acute damage of liver tissue caused by ethanol and drugs. Thus, the pufferfish type I collagen extract has high protective effects on liver.
(4) In the meantime, it is specifically pointed out that another predominant feature of the pufferfish type I collagen extract of the present invention lies in that it can very significantly increase the blood leukocyte and platelet number reduced by chemotherapeutic drugs, increase the weight of immune organ thoracic gland, and increase the body weight, which indicate that the pufferfish type I collagen extract can increase and regulate the immune function of body, improve digestive absorption, enhance body constitution, and reduce the side effects of chemotherapeutic drugs. Hence, the pufferfish type I collagen extract has high effects in immune regulation and health-care.
(5) The pufferfish type I collagen extract of the present invention has many very significant pharmacological activities and health-care effects, pharmacologically acts quickly, and maintains its pharmacological effects for a long-period, i.e., the pufferfish type I collagen extract of the present invention has high performance, quick effects, lasting action and multiple functions.
(6) The pufferfish type I collagen extract of the present invention can significantly improve the digestive function of gastrointestinal tract, regulate gastric dynamics, promote digestion and absorption, maintain the normal digestive function, and thereby significantly increase the body weight of animals in growing period and promote their growth. Further, it can significantly inhibit gastric-emptying, thereby inhibit gastrospam, abdominal pain, diarrhea. These pharmacological activities and health-care effects of the pufferfish type I collagen extract of the present invention are newly disclosed and have never been reported in any document.
(7) The entire process of the present invention for the production of pufferfish type I collagen extract, except for the method c) in the step 2) of claim 4 that belongs to the prior art, is newly applied to the extraction of pufferfish type I collagen extract, has not been reported in any document, and is different from all processes in the prior art for the production of pufferfish glue and pufferfish collagen. Further, although the method c) in the step 2) of claim 4 is a technology of the prior art, it is almost not applied for the extraction of pufferfish type I collagen extract.
(8) The process of the present invention for the production of pufferfish type I collagen extract has a relatively short production cycle (usual 2 to 4 days), a high yield, a high pharmacological activity, a low cost, and can be readily carried out, so that it is simple, high performance, feasible, and suitable for industrial production in large scale. The inventors of the present invention had successfully completed 3 pilot scale experiments for the production of pufferfish type I collagen extract according to the process of the present invention.
(9) The pufferfish type I collagen extract of the present invention is free of tetrodotoxin and other toxins, has a high safety, and can be orally administrated for a long period.
(10) The process of the present invention for the production of pufferfish type I collagen extract substantially generates no contaminative wastes.

Thus, the optimal process of the present invention for the production of pufferfish type I collagen extract (nature pufferfish type I collagen extract or denatured pufferfish type I collagen extract and partial hydrolytes thereof) is novel and not disclosed in any document, and can be readily carried out in industrial scale.

The terms “significant” and “very significant” in the present invention are defined according to their statistical meanings in appropriate statistical tests such as t-test, wherein the P value is less than 0.01 or 0.001.

### BRIEF DESCRIPTION OF THE DRAWINGS

FIG. 1: The protein bands of two subunits α1(I) and α2(I) (represented by α1 and α2 in FIG. 1 according to the marker practices) of the pufferfish type I collagen, wherein the pufferfish type I collagen extract was purified by DEAE-Sepharose fastflow chromatography, subjected to isoelectric focusing polyacrylamide gel electrophoresis, and dyed by Coomassie brilliant blue R250, and said two bands can be readily discriminated according to their dyeing strength, because the α1(I) subunits in the pufferfish type I collagen extract is two times of the α2(I) subunits. According to the measured values of pH gradient of the parallel gels that were subjected to electrophoresis simultaneously, the isoelectric points of two subunits α1(I) and α2(I) of the pufferfish type I collagen were calculated as 4.85±0.5 and 6.71±0.5 respectively (see Example 1).

FIG. 2: The 3.5% SDS-polyacrylamide gel electrophoresis spectrum of the pufferfish type I collagen extract that was purified by DEAE-Sepharose fastflow chromatography, wherein the electrophoresis spectrum indicates that this product is a typical type I collagen (see Example 10). According to the international marker practices of electrophoresis bands of type I collagen protein, α1 and α2 separately represent the bands of two subunits α1(I) and α2(I) of type I collagen protein, β(β₁ and β₂) represents the bands of α1(I)₂ dimer and α1(I)α2(I) dimer, and γ represents the band of [α1(I)]₂α2(I) trimer. The electrophoresis order spectrum of type I collagen in SDS-polyacrylamide gel is: α2, α1, β and γ. The γ band locates near the gel origin because it has a high molecular weight (usually>300 KDa), the β band usually exhibits only one electrophoresis band, the α1 has a content and strength twice as that of the α2, the electrophoresis positions of α1 and α2 are adjacent because they have similar molecular weight, but α2 locates before α1.

FIG. 3: The ultraviolet absorption scanning spectrum of 0.1 mg/mL pufferfish type I collagen extract in 0.1 mol/L hydrochloric acid. It can be seen that the maximum absorption wavelength is 203±3 nm, while there is no absorption peak in the range from 260 nm to 280 nm and the absorption value in said wavelength range is relatively small (see Example 12).

FIG. 4: The 5% SDS-polyacrylamide gel electrophoresis (PAGE) spectrum of the pufferfish type I collagen extract that was subjected to a controllable partial hydrolysis, wherein the electrophoresis bands exhibit the electrophoresis spectrum features of typical type I collagen (see Example 18).

FIG. 5: Results of the identification test of pufferfish type I collagen extract according to conventional single immunodiffusion method, wherein the concentration and precipitation ring of pufferfish type I collagen are in positive correlation, and the white ring is the specific immunoprecipitation ring formed by diffusing the pufferfish type I collagen and its rabbit antiserum. The control collagens or denatured collagens such as various non-pufferfish skin and colla piscis, gelatin, pig skin, etc. do not form such an immunoprecipitation ring in the said test, which indicates that the said identification test has specificity (see Example 31).

FIG. 6: The results of the immunoprecipitation line of pufferfish type I collagen according to conventional counter immunoelectrophoresis, wherein the white band in the lower half of the figure is the specific immunoprecipitation line formed after the electrophoresis of the pufferfish type I collagen and its rabbit antiserum, while there is no immunoprecipitation line formed in the upper half, the electrophoresis region of control collagens or denatured collagens such as various non-pufferfish skin and colla piscis, gelatin, pig skin, etc., which indicate that this identification test has specificity (see Example 31).

### EMBODIMENTS OF THE INVENTION

Since tetrodotoxin is quickly destroyed and loses its toxicity under alkaline conditions, the optimal method for the detoxification of raw material skin, bone and fins for the production pufferfish type I collagen extract is alkaline solution treatment. Since pufferfish type I collagen extract is collagen and/or denatured collagen polypeptide, the best extraction solvent is water or dilute acid solution, the best drying method is freeze-drying, the best hydrolysis method is controllable enzyme hydrolysis or acid hydrolysis, and the best precipitation method is acetone precipitation method. Best use of the preparation of pufferfish type I collagen extract for oral administration is for treatment and prevention of digestive tract diseases (various gastric ulcer, gastric hemorrhage, alcoholic and drug-induced liver damage, liver fibrosis, duodenal ulcer, acute/chronic gastritis, gastrospam, stomach pain, irritable bowel syndrome, colonitis, gastrointestinal function disorder, gastric dynamic disorder, indigestion and malabsorption), immune function disorder and decrease, leucopenia.

Specific examples: the follows are the specific examples of the present invention, but the present invention is not limited by these examples. If it is not specifically explained, the pufferfish skin, bone (fins) used in the following examples are from pufferfish artificially bred in fresh water.

### EXAMPLE 1

500 ml of water was added to 100 g of pufferfish skin and heated to 100° C., and the extraction was performed for 8 hours. After homogenization, the homogenate was clarified by high speed centrifugation, and NaCl was added to the supernatant to reach a final concentration of 0.2 mol/L. Then, the supernatant was loaded to DEAE-Sepharose fastflow column and was eluted by 0.2 mol/L NaCl solution, the eluent was desalted and concentrated by ultrafiltration, and the concentrated solution was spray-dried to obtain a light yellow pufferfish type I collagen extract powder. The isoelectric points of two subunits of pufferfish type I collagen protein were separately α1(I): 4.85±0.5 and α2(I): 6.71±0.5 (see FIG. 1) as measured by isoelectric focusing polyacrylamide gel electrophoresis method. The protein-bound carbohydrate of the pufferfish type I collagen extract was 1.16% according to o-toluidine method after trifluoroacetic acid hydrolysis.

### EXAMPLE 2

1500 ml of water was added to 500 g of pufferfish bone (fins) and heated to 100° C., and the extraction was performed for 10 hours. After filtration, the filtrate was vacuum concentrated to 200 ml, HCl was added to reach a final concentration of 0.2 mol/L, and the hydrolysis was performed at 60° C. for 2 hours. The hydrolysis solution was concentrated and deacidified, and clarified by centrifugation. The centrifugation supernatant was adjusted to have a pH value of 7.4, and NaCl was added to reach a concentration of 0.18 mol/L. Then, the supernatant was loaded to DEAE-cellulose column and was eluted by 0.2 mol/L NaCl solution, the eluent was desalted and concentrated by ultrafiltration, and spray-dried to obtain 47.2 g of a light yellow dry pufferfish type I collagen extract powder.

### EXAMPLE 3

1000 ml of water was added to 100 g of pufferfish skin and heated, and the extraction was performed at 110° C. and 2 atms for 60 minutes. After homogenization, the homogenate was vacuum concentrated to 150 ml, glacial acetic acid was added to reach a final concentration of 0.8 mol/L, and the hydrolysis was carried out at 45° C. for 4 hours. Then, the hydrolysis solution was clarified by centrifugation, and spray-dried to obtain 23 g of a light yellow pufferfish type I collagen extract dry powder. It had a collagen content of 75.92% according to Kivirikko method, and its total protein content was 86.22% according to Kjeldahl determination.

### EXAMPLE 4

2000 ml of water was added to 500 g of pufferfish bone (fins) and 100 g of pufferfish skin, and the extraction was performed at 125° C. and 3 atms for 2 hours. After filtration, the filtrate was stored for standby, 1000 ml of water was added to the filter residue, the same extraction was performed again, and these steps were repeated for 3 times. All filtrates were combined and vacuum concentrated to 200 ml, HCl was added to the concentrated solution to reach a final concentration of 0.01 mol/L, and the hydrolysis was performed at 75° C. for 8 hours. The hydrolysis solution was clarified by centrifugation, and the centrifugation supernatant was dried by spray-drying to obtain 46 g of a light yellow pufferfish type I collagen extract dry powder.

### EXAMPLE 5

500 ml of water was added to 100 g of pufferfish skin, and the extraction was performed at 100° C. for 5 hours. After filtration, the filtrate was stored for standby, 250 ml of water was added to the filter residue, the same extraction was performed again, and these steps were repeated for 2 times. All filtrates were combined and used to homogenate the filter residue, and the homogenate was clarified by centrifugation and was loaded on a DEAE-52 cellulose column in the presence of 0.2 mol/L NaCl. The eluent was desalted by ultrafiltration and concentrated to 150 ml, acetone of 10 times volume was added, and the precipitation was performed at 4° C. overnight. After filtration, a gray precipitate of pufferfish type I collagen extract was obtained, and 21.5 g product was obtained by drying. Said product had a protein-bound carbohydrate content of 1.12%.

### EXAMPLE 6

5000 ml of 0.01 mol/L NaOH was added to 500 g of nature pufferfish skin (toxic), and the immersion was performed at 4° C. for 24 hours. After the immersion alkaline solution was decanted, NaOH was added again and the same immersion was performed for 5 times. The residual alkaline solution was fully washed away by distilled water. 3500 ml of 0.1 mol/L hydrochloric acid was added and the immersion was performed for 48 hours. After homogenization, the homogenate was centrifuged, and the centrifugation supernatant was freeze-dried to obtain 106.6 g of a light white pufferfish type I collagen extract freeze-dried powder.

### EXAMPLE 7

1000 ml of 0.05 mol/L hydrochloric acid was added to 120 g of pufferfish skin. After the pufferfish skin was immersed at 50° C. for 12 hours, the acid solution was poured into a container with 370 g of pufferfish bone (fins), and the immersion was performed at 50° C. for 48 hours. After homogenization, the homogenate was centrifuged, and the centrifugation supernatant was added to the pufferfish skin that had been immersed in hydrochloric acid. After homogenization, the homogenate was vacuum concentrated and deacidified at 70° C. The concentrated solution was centrifuged, the centrifugation supernatant was adjusted to have a pH value of 7.4, and NaCl was added to reach a concentration of 0.18 mol/L. The supernatant was loaded to DEAE-cellulose column, and was eluted by 0.2 mol/NaCl solution. The eluent was desalted and concentrated by ultrafiltration, and was spray-dried to obtain 53.5 g of a light while pufferfish type I collagen extract dry powder.

### EXAMPLE 8

600 ml of 0.5 mol/L acetic acid was added to 50 g of pufferfish, the immersion was performed at 10° C. or below for 72 hours, and pigments in surface layer was removed during the immersion period. After homogenization, the homogenate was centrifuged, and the centrifugation supernatant was freeze-dried to obtain 11 g of a light white pufferfish type I collagen extract freeze-dried powder having amino acid components as shown in Table 1.

TABLE 1
Analysis results of amino acid components of pufferfish
type I collagen extract freeze-dried powder

| | | | Weight |
|---|---|---|---|
| | Weight percentage | | percentage |
| Amino acid | content (%) | Amino acid | content (%) |
| Aspartic acid | 7.12 | Methionine | 1.34 |
| Threonine | 3.47 | Isoleucine | 1.10 |
| Serine | 3.83 | Leucine | 2.43 |
| Glutamic acid | 8.70 | Tyrosine | 0.24 |
| Proline | 15.89 | Phenylalanine | 2.62 |
| Glycine | 15.40 | Lysine | 4.81 |
| Alanine | 11.08 | Histidine | 0.97 |
| Cystine | 0.55 | Arginine | 10.16 |
| Valine | 2.98 | Hydroxyproline | 7.30 |

### EXAMPLE 9

2000 ml of 0.1 mol/L NaOH was added to 500 g of nature pufferfish skin (toxic), and the immersion was performed at 4° C. for 12 hours. After the immersion alkaline solution was decanted, NaOH was added again and the same immersion was performed for 4 times. The residual alkaline solution was fully washed away by distilled water. 7500 ml of 0.5 mol/L acetic acid was added and the immersion was performed for 48 hours. After homogenization, the homogenate was centrifuged, the centrifugation supernatant was adjusted to have a pH of 7.5, NaCl was added to reach a final concentration of 2.5 mol/L. After precipitation at 10° C. or below for 36 hours, a precipitate was obtained by centrifugation. The precipitate was redissolved in 0.1 mol/L acetic acid, desalted by ultrafiltration, and dried by freeze-dry to obtain 119.5 g of a white pufferfish type I collagen extract powder.

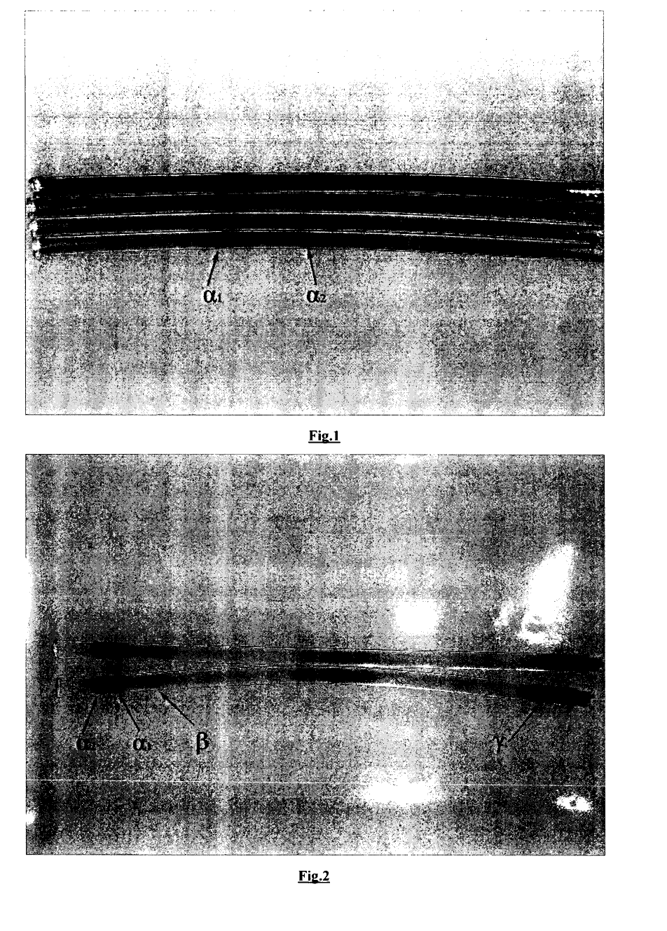

The 3.5% SDS-polyacrylamide gel electrophoresis spectrum of the product indicated that it was a typical type I collagen (see FIG. 2). The pufferfish type I collagen extract had a total protein content of 92% according to Kjeldahl determination and Lowry method, and a collagen protein content of 83% according to Kivirikko method.

### EXAMPLE 10

800 ml of 0.1 mol/L hydrochloric acid was added to 100 g of pufferfish skin, and the immersion was performed at 0° C. for 48 hours. After homogenization, the homogenate was centrifuged, NaCl was added to the centrifugation supernatant to reach a final concentration of 1.0 mol/L, and the supernatant was placed at 4° C. for 24 hours for precipitation. After 11,000×g centrifugation, the precipitate was redissolved in 0.2 mol/L acetic acid, fully dialyzed by 0.2 mol/L NaCl solution having a pH of 7.5, and then was loaded to DEAE-Sepharose fastflow column and eluted by 0.2 mol/L NaCl solution. The eluent with absorption at 230 nm was desalted and freeze-dried to obtain a white pufferfish type I collagen extract freeze-dried powder. Aanalyzed by an automatic amino acid analyzer, the amino acid components of said pufferfish type I collagen extract were shown in Table 2. The collagen protein content of the product is 66.7%, which was calculated based on that the average upper limit of weight percentage of hydroxyproline in fish collagen was 10%.

TABLE 2
Analysis results of amino acid components of pufferfish
type I collagen extract

| | | | Weight |
|---|---|---|---|
| | Weight percentage | | percentage |
| Amino acid | content (%) | Amino acid | content (%) |
| Aspartic acid | 6.67 | Methionine | 1.33 |
| Threonine | 2.67 | Isoleucine | 1.33 |
| Serine | 4.00 | Leucine | 2.67 |
| Glutamic acid | 8.00 | Tyrosine | 0.00 |
| Proline | 10.67 | Phenylalanine | 2.67 |
| Glycine | 24.00 | Lysine | 4.00 |
| Alanine | 10.67 | Histidine | 1.33 |
| Cystine | 1.33 | Arginine | 9.33 |
| Valine | 2.67 | Hydroxyproline | 6.67 |

### EXAMPLE 11

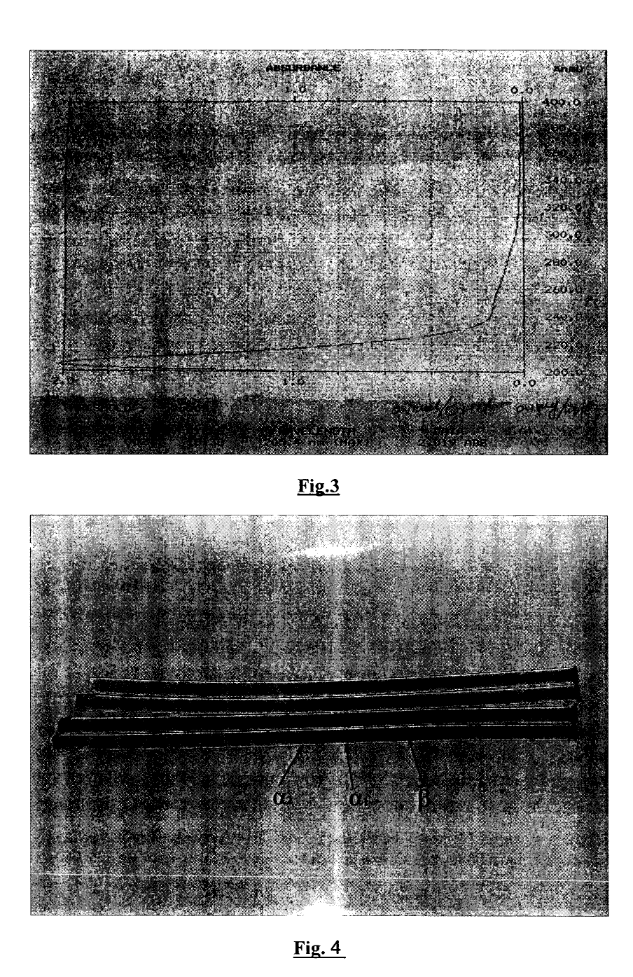

2500 ml of deionized water was added to 500 g of pufferfish bone (fins) and 200 g of pufferfish skin, and the extraction was performed at 95° C. for 2 hours. After filtration, the filtrate was stored for standby, the filter residue was extracted by 2500 mo of water according to the same way, and these steps were repeated for 4 times. All filtrates were combined and vacuum concentrated to 350 ml, and HCl was added to the concentrated solution to reach a final concentration of 0.1 mol/L. After hydrolysis at 55° C. for 2 hours, acetone with 10 times volume was added slowly to the hydrolysis solution under stirring and placed at 4° C. for 48 hours, and a precipitate of pufferfish type I collagen extract was obtained by filtration. The precipitate was dried and pulverized to obtain 89.1 g of a light yellow pufferfish type I collagen extract dry powder having an ultraviolet absorption scanning spectrum as shown in FIG. 3.

### EXAMPLE 12

1200 ml of 0.5 mol/L acetic acid was added to 100 g of pufferfish skin, and the immersion was performed at 10° C. or below for 48 hours. After homogenization, the homogenate was placed for 24 hours and then was centrifuged, the centrifugation supernatant was adjusted to have a pH of 7.4, NaCl was added to reach 0.2 mol/L. The supernatant was loaded on DEAE-cellulose column and was eluted by 0.2 mol/L NaCl solution, NaCl was added to the eluent to reach a final concentration of 2.4 mol/L. The precipitation was performed at 4° C. for 48 hours, and a precipitate was obtained by centrifugation. The precipitate was redissolved in 0.5 mol/L acetic acid, desalted by ultrafiltration, and freeze-dried to obtain 18.4 g of a white ufferfish type I collagen extract freeze-dried powder. Its amino acid components were analyzed by an automatic amino acid analyzer and the results were shown in Table 3.

TABLE 3
Analysis results of amino acid components of pufferfish
type I collagen extract freeze-dried powder

| | | | Weight |
|---|---|---|---|
| | Weight percentage | | percentage |
| Amino acid | content (%) | Amino acid | content (%) |
| Aspartic acid | 6.54 | Methionine | 1.25 |
| Threonine | 3.43 | Isoleucine | 0.93 |
| Serine | 3.43 | Leucine | 2.49 |
| Glutamic acid | 9.66 | Tyrosine | 0.62 |
| Proline | 14.33 | Phenylalanine | 2.18 |
| Glycine | 20.25 | Lysine | 4.36 |
| Alanine | 10.90 | Histidine | 1.25 |
| Cystine | 0.62 | Arginine | 9.35 |
| Valine | 2.49 | Hydroxyproline | 6.23 |

An amount of said pufferfish type I collagen extract freeze-dried powder was redissolved in 0.01 mol/L acetic acid, and the solution was adjusted to have a pH of 7.4 and divided into 3 parts. The first part was hydrolyzed at 37° C. for 3 hours by type I collagenase in the presence of 0.2 mol/L NaCl and 0.01 mol/L CaCl₂, the enzyme was inactivated by heating to 100° C. for 3 minutes, and the solution was stored for standby. The second part was treated by the same way, wherein type I collagenase and a final concentration of 0.01 mol/L EDTA were added, while 0.2 mol/L NaCl and 0.01 mol/L CaCl₂ were not added (EDTA was able to inhibit the activity of type I collagenase). The third part was heat treated by the same way, but no reagent was added. 40 SD rats were divided into 4 groups, and the protection effects of the treated extracts on ethanol-induced rat gastric mucosa damage were observed according to the methods in “Collection of Guidelines for Preclinical Studies of New Drugs (Western Medicines)” edited by China Ministry of Health. The results shown that the pufferfish type I collagen extract exhibit complete protection effects on ethanol-induced rat gastric mucosa damage, but its protection effects were quickly reduced by type I collagenase, and the inhibition of type I collagenase activity could eliminate such reduction. The results were shown in Table 4.

TABLE 4
Protection effects of pufferfish type I collagen extract on ethanol-induced
rat gastric mucosa damage, and the reducing effects of type I collagenase
on such protection effects ( x̅ ± SD), NS: normal saline

| | | | Admin- | |
|---|---|---|---|---|
| | | Animal | istration | |
| Groups | Dose | number | route | Ulcer index |
| Pathologic model | NS | 10 | ig | 123.3 ± 15.6 |
| First part of pufferfish | 0.5 g/kg | 10 | ig | 67.7 ± 6.1 |
| type I collagen extract | | | | |
| Second part of pufferfish | 0.5 g/kg | 10 | ig | 0.5 ± 0.2 |
| type I collagen extract | | | | |
| Third part of pufferfish | 0.5 g/kg | 10 | ig | 0 ± 0 |
| type I collagen extract | | | | |

### EXAMPLE 13

2500 ml of deionized water was added to 500 g of pufferfish bone (fins), and the extraction was performed at 100° C. for 10 hours. After filtration, the filter residue was discarded, 200 g of pufferfish skin was added to the filtrate, and the extraction was performed at 100° C. for 8 hours. After homogenization, the homogenate was concentrated to 300 ml and was adjusted to have a pH of 7.4, type III collagenase was added to 10 mg/100 g of wet skin, NaCl was added to 0.2 mol/L, and CaCl₂ was added to reach a final concentration of 0.01 mol/L. After hydrolysis at 37° C. for 6 hours, the enzyme was inactivated by heating to 100° C. The hydrolysis solution was vacuum concentrated to 200 ml, and spray-dried to obtain 87.5 g of a light yellow pufferfish type I collagen extract dry powder.

### EXAMPLE 14

1500 ml of 0.5 mol/L hydrochloric acid was added to 50 g of natural pufferfish skin (toxic) and treated at room temperature for 6 hours, the immersion solution was decanted, the skin was washed with water, and these steps were repeated for 4 times, total 5 times. The residue acid solution was fully washed away by water, 2000 ml of 0.5 mol/L acetic acid was added. After homogenization, the homogenate was centrifuged, and the centrifugation supernatant was spray-dried to obtain 33.2 g of a white pufferfish type I collagen extract dry powder.

### EXAMPLE 15

1200 ml of 0.5 mol/L acetic acid was added to 100 g of pufferfish skin, and the immersion was performed at 10° C. or below for 48 hours. After homogenization, the homogenate was placed at 10° C. or below for 24 hours, and centrifuged. The centrifugation supernatant was loaded on a CM-cellulose column and eluted by 0-0.2 mol/L NaCl solution in gradient manner, and the eluent having absorption at 230 nm was collected. After the eluent was desalted and concentrated by ultrafiltration, acetone of 10 times volume was added, and the precipitation was performed at 10° C. or below for 24 hours. A precipitate was obtained by filtration, and was dried to obtain 24 g of a light white pufferfish type I collagen extract.

### EXAMPLE 16

2000 ml of deionized water was added to 500 g of pufferfish bone (fins), and the extraction was performed at 100° C. for 10 hours. After filtration, the filtrate was vacuum concentrated to 600 ml. 120 g of pufferfish skin was added to the concentrated solution, and the extraction was performed at 100° C. for 8 hours. After homogenization, acetic acid was added to the homogenate to reach a final concentration of 0.5 mol/L. The hydrolysis was performed at 60° C. for 6 hours. The hydrolysis solution was centrifuged, and the centrifugation supernatant was vacuum concentrated to 200 ml and spray-dried to obtain a pufferfish type I collagen extract dry powder. The electrophoresis bands of the pufferfish type I collagen extract exhibited features of typical type I collagen according to 5% SDS-PAGE electrophoresis test (see FIG. 4). The results of amino acid components of said pufferfish type I collagen extract were shown in Table 5. The pufferfish type I collagen extract had a protein-bound carbohydrate content of 1.8% according to o-toluidine method after trifluoroacetic acid hydrolysis, a total protein content of 80.5% according to Kjeldahl determination, and a collagen protein content of 55.9%.

TABLE 5
Analysis results of amino acid components of pufferfish
type I collagen extract

| | | | Weight |
|---|---|---|---|
| | Weight percentage | | percentage |
| Amino acid | content (%) | Amino acid | content (%) |
| Aspartic acid | 4.99 | Methionine | 1.74 |
| Threonine | 1.75 | Isoleucine | 1.06 |
| Serine | 2.07 | Leucine | 2.21 |
| Glutamic acid | 9.44 | Tyrosine | 0.60 |
| Proline | 9.21 | Phenylalanine | 1.70 |
| Glycine | 19.41 | Lysine | 3.01 |
| Alanine | 7.85 | Histidine | 0.83 |
| Cystine | 0.20 | Arginine | 6.15 |
| Valine | 2.32 | Hydroxyproline | 5.59 |

### EXAMPLE 17

2500 ml of deionized water was added to 500 g of pufferfish bone (fins), the extraction was performed at 100° C. for 5 hours. After filtration, the filtrate was stored for standby, 2500 ml of deionized water was added to the filter residue, the extraction was performed by the same way, and these steps were repeated for total 3 times. All filtrates were combined and vacuum concentrated to 1500 ml. 200 g of pufferfish skin was added to the concentrated solution, and the extraction was performed at 95° C. for 5 hours. After homogenization, a concentrated hydrochloric acid was added to the homogenate to reach a final concentration of 0.05 mol/L, and the hydrolysis was performed at 50° C. under sealing condition for 24 hours. After filtration, the filtrate was vacuum concentrated to 200 ml and centrifuged. The centrifugation supernatant was adjusted to have a pH of 7.4, NaCl was added to reach a concentration of 0.2 mol/L. The supernatant was loaded on DEAE-cellulose column and eluted by 0.2 mol/L NaCl, and the eluent having absorption at 230 nm was collected. The eluent was desalted and concentrated by ultrafiltration, and spray-dried to obtain 82.3 g of a light yellow pufferfish type I collagen extract dry powder.

### EXAMPLE 18

3000 ml of deionized water was added to 250 g of pufferfish skin, the extraction was Performed at 95° C. for 10 hours. After filtration, the filtrate was stored for standby, 2000 ml of deionized water was added to the filter residue, and the extraction was performed by the same way for 10 hours. The extract and the residue were homogenated, and the homogenate was centrifuged and filtered. All filtrates were combined and vacuum concentrated at 80° C. to 450 ml. Hydrochloric acid was added to the concentrated solution to reach a pH of 3, and pepsin was added to reach 50 mg/100 g of wet skin. The hydrolysis was performed at 35° C. under sealing condition for 48 hours, and the enzyme was inactivated by heating to 100° C. for 5 minutes. After centrifugation, the centrifugation supernatant was adjusted to have a pH of 7.4, NaCl was added to 0.2 mol/L. The supernatant was loaded on DEAE-cellulose column, and was eluted by 0.2 mol/L NaCl solution. The eluent was desalted by ultrafiltration, and spray-dried to obtain 53.3 g of a light yellow product.

### EXAMPLE 19

### Protection Effects of Pufferfish Type I Collagen Extract on Anhydrous Ethanol-Induced Gastric Mucosa Damage

60 SD rats with equal numbers of female and male were provided by the Experimental Animal Center of Nanjing Medical University, and the tests were conducted according to the methods in “Collection of Guidelines for Preclinical Studies of New Drugs (Western Medicines)” edited by China Ministry of Health. The results were shown in Table 6.

TABLE 6
Protection effects of pufferfish type I collagen extract on anhydrous
ethanol-induced rat gastric mucosa damage ( x̅ ± SD)

| | | | | | p vs |
|---|---|---|---|---|---|
| | | Animal | | | pathologic |
| Groups | Dose | number | Route | Ulcer index | group |
| Normal control | NS | 10 | ig | 7.0 ± 5.2 | <0.001 |
| Pathologic model | NS | 10 | ig | 120.9 ± 22.6 | |
| Bismuth | 100 mg/kg | 10 | ig | 15.0 ± 6.0 | <0.001 |
| Potassium | | | | | |
| Citrate | | | | | |
| Pufferfish type I | High dose | 10 | ig | 0 ± 0 | <0.001 |
| collagen extract | Middle | 10 | ig | 4.6 ± 4.5 | <0.001 |
| | dose | | | | |
| | Low dose | 10 | ig | 15.0 ± 4.2 | <0.001 |

The results showed that the pufferfish type I collagen extract as prepared in Example 8 exhibit very significant protection effects on anhydrous ethanol-induced rat gastric mucosa damage.

### EXAMPLE 20

### Effects of Pufferfish Type I Collagen Extract on Shay at Gastric Ulcer

50 SD rats with a body weight of 180 to 200 g and equal numbers of female and male were provided by the Experimental Animal Center of Nanjing Medical University, and the tests were conducted according to the methods in “Collection of Guidelines for Preclinical Studies of New Drugs (Western Medicines)” edited by China Ministry of Health.

TABLE 7
Prevention effects of pufferfish type I collagen extract on
pylorus-ligated Shay rat gastric ulcer ( x̅ ± SD)

| | | | | | p vs |
|---|---|---|---|---|---|
| | | Animal | | | pathologic |
| Groups | Dose | number | Route | Ulcer index | group |
| Pathologic model | NS | 10 | ig | 20.3 ± 1.1 | |
| Cimetidine | 80 mg/kg | 10 | ig | 15.8 ± 1.5 | <0.05 |
| Pufferfish type I | High dose | 10 | ig | 1.7 ± 1.1 | <0.001 |
| collagen extract | Middle | 10 | ig | 2.7 ± 2.5 | <0.001 |
| | dose | | | | |
| | Low dose | 10 | ig | 3.6 ± 2.7 | <0.001 |

The results showed that the pufferfish type I collagen extract as prepared in Example 11 exhibit dose-dependently very significant prevention effects on Shay rat gastric ulcer, which indicated that it has significant prevention and treatment effects on digestive gastric ulcer.

### EXAMPLE 21

### Effects of Pufferfish Type I Collagen Extract on Rat Alcoholic Fatty Liver

40 SD male rats with a body weight of 150 to 200 g were provided by the Experimental Animal Center of Nanjing Medical University, and the tests were conducted according to the Methods in “Drug Discovery and Evaluation—Pharmacological Assays” (H. G. Vogel, W. H. Vogel. Chinese version, translated by Du Guanhua, published by Science Publishing House) and related references. The results were shown in Table 8.

TABLE 8
Effects of pufferfish type I collagen extract
on rat alcoholic fatty liver ( x̅ ± SD),
**P < 0.01, *P < 0.05 vs normal control

| | | Animal | Hepatic collagen | Gastric collagen |
|---|---|---|---|---|
| Groups | Dose | number | content (%) | content (%) |
| Normal control | NS | 10 | 0.14 ± 0.04% | 1.29 ± 0.4% |
| Pathologic group | NS | 10 | 0.25 ± 0.09%** | 2.00 ± 0.63%* |
| Pufferfish type I | High | 10 | 0.20 ± 0.06%* | 1.40 ± 0.48%* |
| collagen extract | dose | | | |
| | Low | 10 | 0.19 ± 0.08% | 1.68 ± 0.38% |
| | dose | | | |

The results showed that the pufferfish type I collagen extract as prepared in Example 11 inhibited dose-dependently the pathologic increase of hepatic and gastric wall collagen contents of rats with alcoholic fatty liver, which indicated that the pufferfish type I collagen extract could inhibit the pathologic synthesis of collagen in liver and gastric tissues.

### EXAMPLE 22

### Effects of Pufferfish Type I Collagen Extract on Acetic Acid-Burn Induced Gastric Ulcer in Rats

50 SD rats with a body weight of 180 to 200 g and equal numbers of female and male were provided by the Experimental Animal Center of Nanjing Medical University, and the tests were conducted according to the methods in “Collection of Guidelines for Preclinical Studies Of New Drugs (Western Medicines)” edited by China Ministry of Health.

TABLE 9
Treatment effects of pufferfish type I collagen extract on
acetic acid-burn induced gastric ulcer in rats ( x̅ ± SD)

| | | | | | p vs |
|---|---|---|---|---|---|
| | | Animal | | Ulcer area | pathologic |
| Groups | Dose | number | Route | (mm²) | group |
| Pathologic model | NS | 10 | ig | 70.1 ± 34.4 | |
| Cimetidine | 80 mg/kg | 10 | ig | 44.7 ± 38.4 | <0.001 |
| Pufferfish type I | High dose | 10 | ig | 12.6 ± 10.6 | <0.001 |
| collagen extract | Middle | 10 | ig | 17.9 ± 12.2 | <0.001 |
| | dose | | | | |
| | Low dose | 10 | ig | 15.0 ± 10.1 | <0.001 |

The results showed that the pufferfish type I collagen extract exhibited dose-dependently very significant treatment effects on acetic acid-burn induced gastric ulcer in rats, which indicated that it had treatment effects on chronic gastric ulcer and could significantly promote the healing of ulcer portions.

### EXAMPLE 23

### Effects of Pufferfish Type I Collagen Extract on Cyclophosphamide-Induced Mouse Leukopenia

75 Kunming mice with a body weight of 18 to 22 g and equal numbers of male and female used, and the tests were conducted according to the methods in “Collection of Guidelines for Preclinical Studies of New Drugs (Western Medicines)” edited by China Ministry of Health.

TABLE 10
Effects of pufferfish type I collagen extract on cyclophosphamide-induced
mouse white blood cell (WBC) number reduction ( x̅ ± SD)

| | | Animal | | WBC |
|---|---|---|---|---|
| Groups | Dose | number | Route | (×10⁹/L) |
| Normal control | NS | 15 | ig | 5.3 ± 1.1** |
| Pathologic | 100 mg | 15 | ig | 3.3 ± 1.7 |
| group | cyclophosphamide/kg | | | |
| Pufferfish | High dose | 15 | ig | 6.3 ± 2.0** |
| type I collagen | Middle dose | 15 | ig | 4.2 ± 1.9 |
| extract | Low dose | 15 | ig | 3.6 ± 1.4^{##} |

**P < 0.01, vs pathologic group,
^{##}P < 0.01, vs control group

The results showed that the pufferfish type I collagen extract elevated dose-dependently the mouse white blood cell (WBC) number that had reduced by cyclophosphamide, which indicated that it could enhance immunity and reduce side-effects of chemotherapeutic drugs.

### EXAMPLE 24

### Effects of pufferfish Type I Collagen Extract on PGE₂ Content in Gastric Mucosa of Rat with Indomethacin-Induced Gastric Ulcer

60 SD rats with a body weight of 180 to 200 g and equal numbers of male and female were used, Indomethacin was commercially available from Sigma Company. The PGE₂ content was measured by radioimmunoassay. The tests were conducted according to the methods in “Drug Discovery and Evaluation—Pharmacological Assays” (H. G. Vogel, W. H. Vogel. Chinese version, translated by Du Guanhua, published by Science Publishing House). The results were shown in Table 11.

TABLE 11
Effects of pufferfish type I collagen extract on PGE₂ content
in gastric mucosa of rat with indomethacin-induced
gastric ulcer ( x̅ ± SD), vs pathologic group

| | | Animal | PGE₂ |
|---|---|---|---|
| Groups | Dose | number | (pg/mg protein) |
| Normal control | NS | 10 | 176 ± 79** |
| Pathologic model | NS | 10 | 79 ± 16 |
| Bismuth potassium citrate | 100 mg/kg | 10 | 170 ± 104* |
| Pufferfish type I collagen extract | High dose | 10 | 198 ± 141* |
| | Middle dose | 10 | 140 ± 63 |
| | Low dose | 10 | 138 ± 91 |

The results showed that the pufferfish type I collagen extract elevated dose-dependently the PGE₂ content, which indicated that the pufferfish type I collagen extract could protect and maintain PGE₂ level and blood flow of gastric mucosa, and this is one of mechanisms for treatment of ulcer and protection of mucosa cells.

### EXAMPLE 25

### Time-Pharmacodynamic Tests of Pufferfish Type I Collagen Extract in Body of Rat

50 SD rats with equal numbers of male and female were used. The time-pharmacodynamic of the drug in body of rats with ethanol-induced ulcer was observed. The results showed that the maximum of pharmacodynamic was reached after 30 minutes of the administration of pufferfish type I collagen extract, which indicated that the pufferfish type I collagen extract had quick effect; while 77.78% of the maximum pharmacodynamic was maintained after 18 hours of the administration, which indicated that the pufferfish type I collagen extract has lasting action.

### EXAMPLE 26

### Effects of Pufferfish Type I Collagen Extract on Rat Duodenal Ulcer

50 male SD rats with a body weight of 250 to 300 g were used, and cysteamine was commercially available from Sigma Company. The tests were conducted according to the methods in “Drug Discovery and Evaluation—Pharmacological Assays” (H. G. Vogel, W. H. Vogel. Chinese version, translated by Du Guanhua, published by Science Publishing House).

The results were shown in Table 12, and indicated that the pufferfish type I collagen extract exhibited very significant prevention effects on cysteamine-induced rat duodenal ulcer.

TABLE 12
Treatment effects of pufferfish type I collagen extract on
cysteamine-induced rat duodenal ulcer ( x̅ ± SD)

| | | Animal | Ulcer | Inhibition |
|---|---|---|---|---|
| Groups | Dose | number | index | ratio (%) |
| Pathologic model | | 8 | 2.8 ± 0.5 | |
| Famotidine | 20 mg/kg | 7 | 2.0 ± 1.3 | 27.27 |
| Pufferfish type I | High dose | 8 | 1.4 ± 0.9** | 50.00 |
| collagen extract | Middle | 8 | 1.8 ± 1.0* | 36.36 |
| | dose | | | |
| | Low dose | 7 | 1.7 ± 1.4 | 37.66 |

### EXAMPLE 27

### Effects of Pufferfish Type I Collagen Extract on NO Content in Gastric Mucosa of Rat with Ethanol-Induced Gastric Ulcer, iNOS activity and expression levels of iNOS and cNOS Genes

The tests of rats with ethanol-induced gastric ulcer were conducted according to the methods in “Collection of Guidelines for Preclinical Studies of New Drugs (Western Medicines)” edited by China Ministry of Health. The effects of pufferfish type I collagen extract on NO content in rat gastric mucosa, iNOS activity and expression levels of iNOS and cNOS genes were observed. NOSs mRNA was extracted by Trizol kit and amplified by RT-PCR. The results showed that, on the one hand, the pufferfish type I collagen extract very significantly reduced dose-independently the NO level in rat gastric mucosa with ethanol-induced damage, the iNOS activity, and the expression level of iNOS gene, very significantly reduced the NO content and iNOS activity below normal level; on the other hand, the pufferfish type I collagen extract very significantly elevated the expression level of cNOS gene that had been reduced by ethanol and the expression of cNOS gene rebounded to its normal level. These indicated that the pufferfish type I collagen extract could differentially regulate the NO level in gastric mucosa, the iNOS activity, and expression of iNOS and cNOS genes, which was one of important mechanisms for protection of gastric mucosa, treatment of ischemia-induced gastric mucosa damage and necrosis, prevention and treatment of gastrointestinal tumors, prevention and treatment of gastric ulcer.

### EXAMPLE 28

### Long-Term Toxicity Test of Pufferfish Type I Collagen Extract

Beagle dog long-term toxicity test of pufferfish type I collagen extract was performed according to “Technologic Requirements for Pharmacological and Toxicological Studies on New drugs” issued by the China State Drug and Food Administration and relevant methods of ICH. The results showed that the high and middle doses of the pufferfish type I collagen extract significantly increased the body weight of test dogs during the 3 months of long-term toxicity test. The high, middle and low doses of the pufferfish type I collagen extract did not significantly affect the plasma biochemical indexes and blood routine examination indexes of the test dogs. The pufferfish type I collagen extract increased the thoracic gland index of the test animals, while the indexes of other organs of the test dogs were normal. The results indicated that the pufferfish type I collagen extract could be safely and effectively administrated for a long term. The high dose of pufferfish type I collagen extract could increase the body weight of test animals and could enhance the immunity.

### EXAMPLE 29

### Test of Pufferfish Type I Collagen Extract for Inhibiting the Angiogenesis in Chick Embryo (CAM)

The normal growth of chick embryo comprises the formation of external blood vascular system in yolk membrane, and the nutrition transportation from egg yolk via yolk membrane. When the pufferfish type I collagen extract was applied to the yolk membrane, the substances having activity of inhibiting angiogenesis in the extract could inhibit the formation of new vessels in the yolk membrane.

Methylcellulose disks (inert solid and transparent substrate) comprising different amounts of the pufferfish type I collagen extract and control drugs were placed at the exterior margin circumjacent yolk membrane blood vessels, where the angiogenesis occurred. The positive control was methylcellulose disks comprising 1.5 mg/ml 2-methoxyestradiol. The control disks and the pufferfish type I collagen extract disks were placed on yolk membranes of embryos with an age of 3 days. At the sites, only main vessels grew to yolk. In the meantime, the methylcellulose disk comprising negative control or an amount of the pufferfish type I collagen extract were placed on the yolk membrane of the same embryo. Two disks were arranged in an axial symmetric manner relative to the head and tail of embryo, so that the individual difference was reduced when the effects of the pufferfish type I collagen and negative control were evaluated. After the disks were placed for 24 hours, the angiogenesis was evaluated, and the result was expressed as the percentage of embryos whose angiogenesis was affected. As comparing the pufferfish type I collagen extract to the negative control (when many fine vessels had been formed), the routes of angiogenesis showed deflection and attenuation, and it was deemed that the angiogenesis was inhibited when the growth of vessel under disks was not observed. The results indicated that the pufferfish type I collagen extract could significantly block formation of new vessels in chick embryo.

### EXAMPLE 30

### Determination of Toxicity of Tetrodotoxin of Pufferfish Artificially Bred in Fresh Water

Pufferfishes (Fugu obscurus, Fugu rubripes and Fugu flavidus) with a body weight ranging from 50 g to 1250 g during different growth periods (corresponding to an age of from 6 months to 2 years) as artificially bred in fresh water were dissected, and their ovaries, seminal vesicles, livers, skins, blood, bones, meat, hearts, eyeballs and other internal organs were separately cut and homogenated with 0.2 mol/L acetic acid in a ratio of 1:3 (w/v), the homogenates were placed at room temperature for 6 hours under occasionally stirring, and were adjusted by 1 mol/L sodium carbonate to reach a pH of 6-7. Mice were administrated with homogenates of various organs in a dose of 0.8 ml/20 g/once per 8 hours, total 3 times (corresponding to 2.4 kg within 24 hours for a 60 kg human). Each organ homogenate was used to intragastrically administrate 5 mice. The results showed that all mice survived, which proved that pufferfishes artificially bred in fresh water during various growth periods were nontoxic.

### EXAMPLE 31

### Counter Immunoelectrophoresis and Immunodiffusion of Pufferfish Type I Collagen Extract

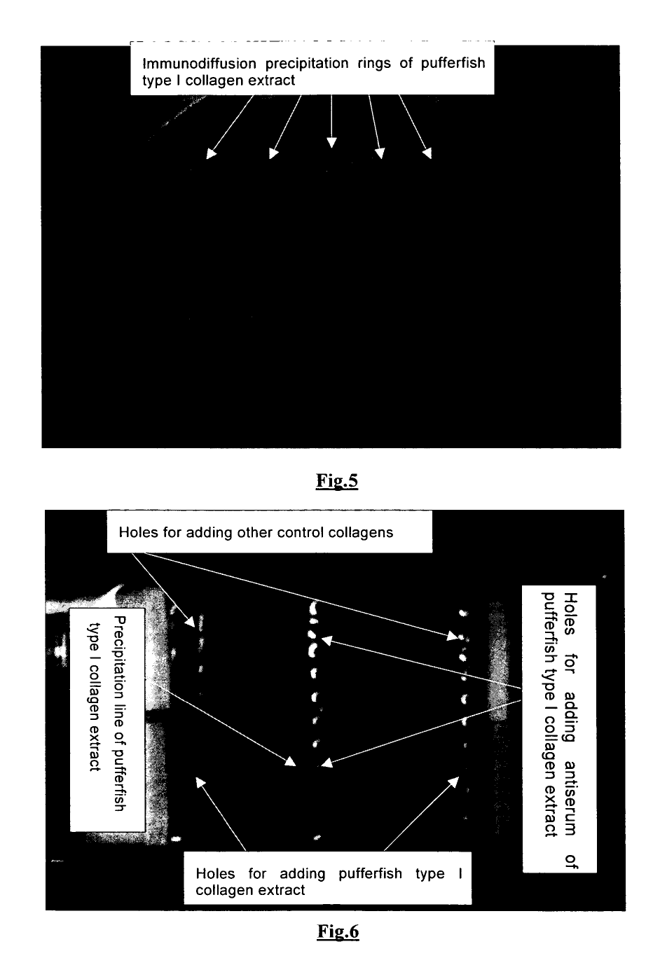

The pufferfish type I collagen extract as prepared in Example 2 was purified by DEAE-Sepharose fastflow, and was used to immunize rabbits in order to obtain rabbit antiserum of pufferfish type I collagen extract. The counter immunoelectrophoresis and immunodiffusion were performed according conventional methods. The immunoprecipitation lines of the pufferfish type I collagen extract were observed and shown in FIG. 5 and FIG. 6. The size of diffusion rings in FIG. 5 was in positive correlation with the concentration of Pufferfish type I collagen extract in sample. The present test can also be used as a quantitative method to determine the content of effective components in pufferfish type I collagen extract. The white band in the lower half of FIG. 6 was the specific immunoprecipitation line formed after the electrophoresis of the pufferfish type I collagen and its rabbit antiserum, while there was no immunoprecipitation line formed in the upper half, the electrophoresis region of the control collagens or denatured collagens such as various non-pufferfish skin, colla piscis, bone, Sigma gelatin, donkey-hide glue, pig skin, etc., which indicated that this identification test has specificity.

### EXAMPLE 32

### Process for the Production of Tablets of Pufferfish Type I Collagen Extract

The process comprises mixing uniformly a pufferfish type I collagen extract as crude drug with microcrystalline cellulose and starch in a certain ratio, granulating via one step, drying, adding an amount of dry starch and talc powder, mixing uniformly, and tabletting.

### EXAMPLE 33

### Process for the Production of Capsules and Enteric Capsules of Pufferfish Type I Collagen Extract

The process comprises mixing uniformly a pufferfish type I collagen extract as crude drug with starch and hydroxylpropylcellulose in a certain ratio, granulating, drying, adding mnagnesium stearate, mixing uniformly, packing into 3# capsules or enteric capsules.

### EXAMPLE 34

### Process for the Production of Chewable Tablets of Pufferfish Type I Collagen Extract

The process comprises mixing uniformly a pufferfish type I collagen extract as crude drug with mannitol in a certain ratio, granulating via one step, blending magnesium stearate in an amount of 0.01 to 0.02 times the weight of pufferfish type I collagen extract dry powder with aromatic spice, adding the blend to granules, mixing uniformly, and tabletting.

### EXAMPLE 35

### Process for the Production of Dissolved Medicine of Pufferfish Type I Collagen Extract

The process comprises adding sugar powder and dextrin to a concentrated solution of pufferfish type I collagen extract to form a soft stuff, granulating, drying, and sub-packaging.

### EXAMPLE 36

### Process for the Production of Drop Pills of Pufferfish Type I Collagen Extract

The process comprises adding an amount of polyethylene glycol-6000 and ethylparaben to a concentrated solution of pufferfish type I collagen extract, heating to 90 to 100° C., sealing and keeping the temperature at 80 to 90° C., adjusting valve to control liquid droplets, dropping into a liquid paraffin, and drying.

### EXAMPLE 37

### Process for the Production of Glue Preparation of Pufferfish Type I Collagen Extract

The process comprises adding an amount of yellow wine, crystal sugar and ethylparaben to a concentrated solution of pufferfish type I collagen extract as prepared in Example 8, continuously concentrating to obtain an ointment, cooling, cutting, and vacuum packing.

### EXAMPLE 38

### Test of Acute Toxicity of Pufferfish Type I Collagen Extract

The test was conducted according to “Technologic Requirements for Pharmacological and Toxicological Studies on New drugs” issued by China State Drug and Food Administration and relevant methods of ICH. The results of pretest indicated that the acute toxicity parameter LD50 were not measurable when mice were intragastrically administrated with the pufferfish type I collagen extracts as prepared in Examples 8, 9, 11, 12 and 16 in their maximum concentrations and maximum doses.

The results of the test indicated that no animal died during 7 days after the administration, which means that the pufferfish type I collagen extracts were highly safe.

### EXAMPLES 39

### Effects of Pufferfish Type I Collagen Extract on Gastric Emptying

105 Kunming mice with a body weight of from 20 to 25 g and equal numbers of male and female were used. The test was conducted according to the methods in “Collection of Guidelines for Preclinical Studies of New Drugs (Western Medicines)” edited by China Ministry of Health, and the phenol red method (Shi G., et al. Gut, 41:612-618, 1997). The results were shown in Table 13. The results indicated that the pufferfish type I collagen extract exhibit dose-dependently very significant inhibition effects on the mouse gastric emptying and the gastric emptying promoted by pyridostigmine bromide, which means that the pufferfish type I collagen extract could block the action of acetylcholine, inhibit the gastric smooth muscle contraction stimulated by acetylcholine, prolong the retention time of food in gastrointestinal tract, and promote the digestion and absorption of food.

TABLE 13
Inhibition effects of pufferfish type I collagen extract on mouse gastric emptying
( x̅ ± SD)

| | | | Amount of | |
|---|---|---|---|---|
| | | | residual phenol | |
| | | Animal | red in stomach | Gastric emptying |
| Groups | Dose | number | (mg) | ratio (%) |
| Standard group | | 15 | 7.61 ± 2.78 | |
| Normal control group | NS | 15 | 1.28 ± 0.75 | 86.0 ± 7.9^{#} |
| Pyridostigmine bromide | 0.1 mg/kg | 15 | 0.47 ± 0.36 | 93.8 ± 4.8* |
| Pufferfish type I collagen | High dose + 0.1 mg/kg | 15 | 1.66 ± 0.17 | 84.0 ± 11.7***^{#} |
| extract + pyridostigmine | | | | |
| bromide | | | | |
| Pufferfish type I collagen | High dose | 15 | 1.86 ± 0.1 | 75.0 ± 13.3***^{#} |
| extract | Middle | 15 | 1.70 ± 0.16 | 77.2 ± 10.1***^{#} |
| | dose | | | |
| | Low dose | 15 | 1.24 ± 0.58 | 83.2 ± 23.2* |

***P < 0.001,
*P < 0.01 vs pyridostigmine bromide group
^{#}P < 0.05 vs normal control group

### EXAMPLE 40

### Process for the Production of Effervescent Tablets of Pufferfish Type I Collagen Extract

The process employs the spray-drying absorption method for the production of microcapsules, and comprises spraying a polyethylene glycol-6000 solution comprising sodium bicarbonate in a coating pot containing a pufferfish type I collagen extract dry powder, sieving to obtain granules; sieving citric acid and aspartame, mixing uniformly with the pufferfish type I collagen extract granules and fumaric acid fine powder, and tabletting under the irradiation of infrared light at the inlet. The process is characterized in that sodium bicarbonate is coated by polyethylene glycol-6000 via the method for producing microcapsules; fumaric acid acts as both foaming agent and water-soluble lubricating agent; and the irradiation of infrared light at the inlet could control the granules at an appropriate temperature, increase the soft-moist feature, and further ensure that tablets do not adhere die.

### EXAMPLE 41

### Protection Effects of Pufferfish Type I Collagen Extract on Ethanol-Induced Acute Rat Liver Damage

50 SD rats with a body weight of 180 to 200 g and equal numbers of female and male were used, and the tests were conducted according to the methods in “Collection of Guidelines for Preclinical Studies of New Drugs (Western Medicines)” edited by China Ministry of Health. The blood serum glutamate-pyruvate transaminase and glutamic oxalacetic transaminase activities were determined by an automatic biochemical analyzer. The results were shown in Table 5.

TABLE 5
Protection effects of pufferfish type I collagen extract on
ethanol-induced acute rat liver damage ( x̅ ± SD)

| | | | | Glutamate | glutamic |
|---|---|---|---|---|---|
| | | | | pyruvate | oxalacetic |
| | | Animal | | transaminase | transaminase |
| Groups | Dose | number | Route | (U) | (U) |
| Normal | NS | 10 | ig | 41.0 ± 3.1*** | 156.7 ± 8.1*** |
| group | | | | | |
| Pathologic | NS | 10 | ig | 59.5 ± 5.2 | 221.3 ± 15.6 |
| group | | | | | |
| Pufferfish | High | 10 | ig | 39.0 ± 1.4*** | 162.0 ± 4.2*** |
| type I | dose | | | | |
| collagen | Middle | 10 | ig | 38.7 ± 6.3*** | 156.3 ± 5.7*** |
| extract | dose | | | | |
| | Low | 10 | ig | 49.7 ± 15.0** | 186.0 ± 20.9** |
| | dose | | | | |

***p < 0.001,
**p < 0.01 vs pathologic group

The results showed that the pufferfish type I collagen extract exhibited dose-dependently very significant protection effects on ethanol-induced acute rat liver damage.

### EXAMPLE 41

### Treatment Effects of Pufferfish Type I Collagen Extract on Acetic Acid-Induced Rat Colonitis

The test was conducted according to the method in the document (Ritzpatrick, R. et al., Agents Actions. 1990, 30:393-402). SD rats were subjected to rectoclysis to form models of colonitis. From the 4^{th} day after the models had been made, the rats were intragastrically administrated for 4 days. The results were shown in Table 14 and Table 15.

TABLE 14
Treatment effects of pufferfish type I collagen extract on 10%
acetic acid-induced rat ulcerative colonitis ( x̅ ± SD)

| | | Animal | |
|---|---|---|---|
| Groups | Dose | number | Score of ulcer index |
| Pathologic group | NS | 10 | 9.16 ± 1.32^{##} |
| Control group | NS | 10 | 0.33 ± 0.51***^{#} |
| Famotidine | 10 mg/kg | 10 | 4.14 ± 3.76* |
| Pufferfish type I collagen | High dose | 10 | 3.33 ± 3.66** |
| extract | Middle dose | 10 | 5.14 ± 3.93* |
| | Low dose | 10 | 7.33 ± 3.54 |

***P < 0.001,
**P < 0.01,
*P < 0.05 vs pathologic group;
^{##}P < 0.01,
^{#}P < 0.05 vsfamotidine

TABLE 15
Effects of pufferfish type I collagen extract on bowel-body
ratio of rat with 10% acetic acid-induced colonitis ( x̅ ± SD)

| | | | Ratio of bowel |
|---|---|---|---|
| | | | weight to |
| | | Animal | body weight |
| Groups | Dose | number | (×10⁻²) |
| Pathologic group | NS | 10 | 2.32 ± 1.06 |
| Control group | NS | 10 | 0.51 ± 0.10*** |
| Famotidine | 10 mg/kg | 10 | 0.92 ± 0.45** |
| Pufferfish type I collagen extract | High dose | 10 | 0.69 ± 0.38** |
| | Middle dose | 10 | 1.42 ± 0.99 |
| | Low dose | 10 | 1.69 ± 0.73 |

***P < 0.001,
**P < 0.01,
*P < 0.05 vs pathologic group

The results indicated that the high and middle doses of pufferfish type I collagen extract exhibited significant treatment effects on 10% acetic acid-induced ulcerative colonitis, but normal level was not reached. The high dose of pufferfish type I collagen extract exhibited significant protection effects on the ratio of bowel weight to body weight of rat with acetic acid-induced colonitis,

### EXAMPLE 43

### Treatment Effects of Pufferfish Type I Collagen Extract on Trinitrobenzene Sulfonic Acid (TNBS)-Induced Rat Colonitis

The test was counted according to the method in the document (Morris, C. P. et al: Gastroenterology. 1989, 96:795-803). TNBS was commercially available from Sigma Company. SD rats were subjected to rectoclysis of 100 mg/kg of TNBS to form models of colonitis. Each group included 10 rats. From the 14^{th} day after the models had been made, the rats were intragastrically administrated for 7 days. The rats were killed by drawing their necks and were dissected, and their colon sections were histopathologically observed. The partial results were shown in Table 16.

TABLE 16
Treatment effects of pufferfish type I collagen extract on the body
weight of rat with TNBS-induced ulcerative colonitis ( x̅ ± SD)

| | | Increment of | p vs pathologic |
|---|---|---|---|
| Groups | Dose | body weight | group |
| Normal group | NS | 75.45 ± 15.16 | <0.01 |
| Pathologic model group | NS | 48.27 ± 24.48 | |
| Sulfasalazine | 0.3 g/kg | 68.89 ± 21.64 | <0.05 |
| Pufferfish type I collagen | High dose | 77.22 ± 22.05 | <0.01 |
| extract | Middle dose | 70.25 ± 11.05 | <0.05 |
| | Low dose | 66.22 ± 27.52 | >0.05 |

The results showed that the pufferfish type I collagen extract elevated significantly the body weight of rat with TNBS-induced colonitis, so that the body weight reached to its normal level. The results of colon sections showed that the pufferfish type I collagen extract exhibited significant treatment effects on TNBS-induced rat colonitis.

The present invention is described above. It is understandable that the person skilled in the art has sufficient ability and knowledge to replace some parts of the present invention by analogs or similar methods in the prior art and to achieve the same object with the proviso of not deviating the teaching of the present invention. Thus, these modifications are covered by the present invention obviously.

1. (canceled)

2. The method according to apy one of claims 11-19 wherein the pufferfish type I collagen extract is administered in the form of an oral preparation.

3. The method according to any one of claims 11-19 wherein the pufferfish type I collagen extract is prepared from pufferfish skin and/or pufferfish bone including fins.

4. A process for the production of a pufferfish type I collagen extract from pufferfish skin and/or pufferfish bone including fins, comprising the following steps:
1) pre-treating raw materials:
a) pre-treating natural pufferfish skin and bone raw material to remove toxin: treating the raw material in an acid solution or alkaline solution at 0 to 50° C. for 4 to 48 hours, sufficiently washing with water, and repeating this step for 4 to 6 times; wherein when an alkaline solution is used for the removal of toxin, the preferred conditions are: normal pressure, a final alkaline solution concentration of 0.01 to 0.1 mol/L, 20-30° C., detoxifying for 8 to 24 hours, and repeating for 4 to 5 times; and when an acid solution is used for the removal of toxin, the preferred conditions are: normal pressure, a final acid solution concentration of 0. 1 to 0.2 mol/L, 0-20° C., detoxifying for 6 to 24 hours, and repeating for 4 to 5 times;
b) washing clearly the skin and bone of the pufferfish artificially bred in fresh water or washing the detoxified skin and bone of natural pufferfish with water, and storing at −20° C. or below for standby if it is not used immediately;
2) extracting according to one of the following three methods:
a) adding water or acid solution to the pre-treated raw material of pufferfish skin and bone in any proportion, extracting at a temperature of from 0 to 125° C., a pressure of from normal pressure to 3 atms for 60 minutes to 100 hours, filtering to obtain the liquid portion, repeating for 0 to 6 times, combining the filtrates, adding water to the residue or combining the filtrates with the residue, homogenizing to obtain a homogenate, standing the homogenate obtained by the acid solution as extracting solvent at 20° C. or below for 12 to 48 hours; the homogenate obtained by water as extracting solvent will be directly used in the next step; wherein when the acid solution is used for the extraction, the preferred conditions are: normal pressure, 0 to 10° C., a final acid solution reaction concentration of 0.1 to 0.5 mol/L, extracting for 48 to 72 hours, repeating for 2 to 4 times, homogenizing; and normal pressure, 40 to 80° C., a final acid solution reaction concentration of 0.01 to 0.2 mol/L, extracting for 4 to 8 hours, repeating for 3 to 5 times, and homogenizing; wherein when water is used for the extraction, the preferred conditions are: normal pressure, 90 to 100° C., extracting for 3 to 8 hours, repeating for 1 to 3 times, and homogenizing;
b) adding water or acid solution to the pre-treated pufferfish bone raw material, extracting at a temperature of from 0 to 125° C. and a pressure of from normal pressure to 3 atms for 60 minutes to 100 hours, filtering to obtain the liquid portion, repeating for 0 to 6 times, combining filtrates, discarding residue, concentrating the filtrates to 100% to 10% of the original volume, adding an amount of pufferfish skin raw material, extracting at a temperature of from 0 to 125° C. and a pressure of from normal pressure to 3 atms for 60 minutes to 100 hours, filtering to obtain the liquid portion, adding water or the same acid solution for extraction, and repeating for 0 to 6 times, combining filtrates, combining the filtrates and the residue, homogenizing to obtain a homogenate, standing the homogenate obtained by the acid solution as extracting solvent at 20° C. or below for 12 to 48 hours; the homogenate obtained by water as extracting solvent will be directly used in the next step; wherein when the acid solution is used for the extraction, the preferred conditions are: normal pressure, 0 to 10° C., a final acid solution reaction concentration of 0.1 to 0.5 mol/L, extracting for 48 to 72 hours, repeating for 2 to 4 times, homogenizing; and normal pressure, 40 to 80° C., a final acid solution reaction concentration of 0.01 to 0.2 mol/L, extracting for 4 to 8 hours, repeating for 3 to 5 times, and homogenizing; wherein when water is used for the extraction, the preferred conditions are: normal pressure, 90 to 100° C., extracting for 3 to 8 hours, repeating for 1 to 3 times, and homogenizing;
c) obtaining the pufferfish type I collagen extract of the present invention by the conventional methods or modified methods for extracting type I collagen and gelatin in the prior art;
3) filtrating and concentrating:
centrifuging or filtering the homogenate to remove residue, optionally concentrating the filtrate to 100% to 10% of the original volume to obtain a concentrated pufferfish type I collagen extract; wherein when the extraction is carried out by using acid solution at low temperature, the preferred method for removing residue is high speed centrifugation at a low temperature; while when the extraction is carried out by using water or the acid solution at high temperature, the preferred method for removing residue is filtration; when the extraction is carried out by using acid solution at low temperature, the preferred concentration method is the concentration by ultrafiltration using a ultrafiltration membrane with a pore diameter of 100 to 200 Kda; when the extraction is carried out by using water or acid solution at high temperature, the preferred concentration method is vacuum concentration; the preferred simple production method comprises: after the aforementioned extract is centrifuged or filtrated to remover residue, it is directly subjected to (ultrafiltration) concentration and (freeze, spray) drying to obtain the pufferfish type I collagen extract;
4) optionally, drying and pulverizing:
drying the extract or the concentrated extract (spray-drying, freeze-drying, or microwave-drying, drying by baking, drying in the shade, preferably freeze-drying or spray-drying), pulverizing to obtain a pufferfish type I collagen extract, a light-yellow or white powdery product capable of passing through a 80 mesh sieve; wherein the acid solution is an organic acid or inorganic acid; the alkaline solution is an inorganic alkaline solution; the final concentration in the extraction step is 0.001 to 1.0 mol/L; the final concentration in the detoxification step is 0.01 to 0.5 mol/L; the examples of the used acid are: formic acid, acetic acid, propionic acid, malonic acid, butyric acid, succinic acid, malic acid, citric acid, tartaric acid, lactic acid, phosphoric acid, hydrochloric acid, sulfuric acid, nitric acid; the examples of the used alkali are: sodium hydroxide, potassium hydroxide, calcium hydroxide (lime water), sodium carbonate; the examples of the used enzyme are: trypsin, pancreatin, pepsin, papain, chymotrypsin, bromelain, dispase, pronase, fibrin, gelatinase, type II collagenase, type III collagenase, proteinase K, and various proteinases from other animals, plants and microorganisms.

5. A process according to claim 4, characterized in that a controllable partial hydrolysis step is performed according to one of the following two methods is performed after the filtration and concentration steps:
1) hydrolyzing by using a proteinase under conditions of: a proteinase concentration of 1 to 100 mg/100 g wet weight tissue, preferably 10-50 mg/100 g wet weight tissue in the reaction system, stirring, a temperature of 20-65° C., preferably 30-37° C., a time of 3-100 hours, preferably 3-48 hours, heating to 100° C. for 5-10 minutes to inactivate the enzyme after the end of enzymolysis;
2) hydrolyzing by using an organic acid and/or an inorganic acid under conditions of: an acid concentration of 0.001-1.0 mol/L, preferably 0.05-0.50 mol/L in the reaction system, stirring, a temperature of 0-100° C., preferably 25-75° C., a time of 60 minutes to 72 hours, preferably 3 to 24 hours, neutralizing or removing acid under vacuum;
optionally, concentrating the hydrolysis solution to a volume of 100% to 10% of the original volume, and drying the concentrated hydrolysis solution to obtain a pufferfish type I collagen extract, or precipitating and drying to obtain a pufferfish type I collagen extract; wherein the used acid and enzyme are those defined in claim 4; the preferred enzyme is type III collagenase, trypsin, pepsin; and the preferred acid is acetic acid and hydrochloric acid.

6. A process according to claim 4, characterized in that a sedimentation step is performed according to one of the following two methods after the concentration step:
1) adding to the concentrated extract a cold acetone having a volume of 8 to 15 times, preferably 10 to 12 times the volume of the concentrated extract, sedimentating at 10° C. or below for 24 to 48 hours, centrifuging or filtering to obtain a precipitate, volatilizing the residual organic solvent from the precipitate, and drying to obtain the pufferfish type I collagen extract;
2) adding to the concentrated extract a cold ethanol until the final ethanol concentration reaches 55-90%, preferably 75-90%, sedimentating at 10° C. or below for 24-48 hours, centrifuging or filtering to obtain a precipitate, volatilizing the residual organic solvent from the precipitate, optionally drying to obtain the pufferfish type I collagen extract.

7. A process according to claim 6, characterized in that the obtained precipitate is repetitively extracted by a neutral buffer (pH 7.5) of 1.0 to 2.2 mol/L NaCl or directly by 1.0 to 2.2 mol/L NaCl solution, the extract solution is desalted, and optionally dried to obtain a pufferfish type I collagen extract with a relatively high purity.

8. The process according to claim 4, wherein the concentrated solution obtained in step 3) is neutralized, filtered and desalted, and then is purified by DEAE- and/or CM-ion exchange chromatography to remove other protein impurities, and the eluent of the ion exchange is desalted and dried to obtain a pufferfish type I collagen extract with a high purity.

9. A pufferfish type I collagen extract prepared by a process according to any one of claims 8 and 20-22.

10. A pufferfish type I collagen extract prepared by a process according to any one of claims 4-8 and 20-22, characterized in that the main chemical components and pharmacologically active components of said pufferfish type I collagen extract are nature pufferfish type I collagen or denatured pufferfish type I collagen and partial hydrolytes thereof, and the pufferfish type I collagen extract has the following features:
a) the pufferfish type I collagen extract is prepared by using pufferfish skin and/or bone (fins) as raw materials, comprises pufferfish type I collagen or denatured type I collagen and partial hydrolytes thereof as main chemical components and pharmacologically active components, and has physical/chemical properties of typical (fish) type I collagen;
b) the content of pufferfish type I collagen or denatured pufferfish type I collagen and partial hydrolytes thereof in the pufferfish type I collagen extract is greater than 50%, and the total protein content is greater than 70%;
c) the molecular weight of pufferfish type I collagen protein trimer [α1(I)]₂α2(I)] is from 300 to 420 KDa, and the molecular weight of the denatured pufferfish type I collagen protein (comprising α1(I) monomer, α2(I) monomer, α1(I)₂ dimer and α1(I)α2(I) dimer) and partial hydrolytes thereof is from 60 to 300 KDa; the isoelectric points of the two subunits of the pufferfish type I collagen protein separately are α1(I):4.85±0.5 and α2(I):6.71±0.5 (see FIG. 1) according to isoelectric focusing polyacrylamide gel electrophoresis method, while the isoelectric points of the two subunits of pufferfish type I collagen protein may vary according to the species of pufferfish;
d) the results of the ultraviolet absorption scanning by using Perkin Elmer Lambda 2 UV-Vis Spectrometer show that the maximum wavelength of ultraviolet absorption of 0.3 mg/ml pufferfish type I collagen extract solution obtained by using 0.2 mol/L acetic acid as solvent is 226±3 nm; while the maximum wavelength of ultraviolet absorption of 0.1 mg/ml pufferfish type I collagen extract solution obtained by using 0.1 mol/L hydrochloric acid as solvent is 203±3 nm; and further, there is no absorption peak in the range from 260 to 280 nm and the absorption value in said wavelength range is relatively low (see FIG. 3);
e) According to the measurements of Kivirikko method and automatic amino acid analyzer, the pufferfish type I collagen extract has a weight percentage content of hydroxyproline of greater than 4.5% that is similar to that of other fish collagen, the weight percentage content of hydroxyproline of fish collagen is usually lower than 10%, which is significantly lower than the content of hydroxyproline (14%) in the collagen of terrestrial animal; the amino acid components of the pufferfish type I collagen extract obtained by a process according to any one of claims 4 to 8 are shown in Tables 1, 2, 3 and 5; the pufferfish type I collagen extract is a glycoprotein and has a protein-bound carbohydrate content of 0.5 to 1.9%; and it is understandable that the data difference is caused by different raw materials and different extraction conditions, but they are measured based on pufferfish type I collagen as main chemical component and pharmacologically active component;
f) the pufferfish type I collagen extract is soluble in water and dilute acid solution, wherein the water solution is thermostable and can maintain its pharmacological activity after being heated at 95 to 100° C. for several hours; the dilute weak acid solution of pufferfish type I collagen extract (less than 0.5 mol/L) maintains a substantially stable pharmaceutical activity after being placed at a temperature from −20° C. to room temperature for a long period; however, the pufferfish type I collagen extract is very sensitive for alkali and may fully lose its pharmaceutical activity in a weak alkali solution, even it is merely placed at room temperature for several hours; the pufferfish type I collagen extract is not sensitive for type III collagenase, and is sensitive for type I collagenase; after the pufferfish type I collagen extract is hydrolyzed by a type I collagenase, its pharmacological and biological activity decreases quickly.

11. A method of preventing or treating a gastrointestinal disease comprising administering to a human with a gastrointestinal disease a pufferfish type I collagen extract.

12. The method of claim 11, wherein the gastrointestinal disease is selected from the group consisting of gastric ulcer, alcohol-induced gastric ulcer, drug-induced gastric ulcer, gastrorrhagia, alcohol-induced gastric mucosa injury, drug-induced gastric mucosa injury, stress gastric ulcer, acute gastritis, chronic gastritis, superficial erosive gastritis, erosive gastritis, gastrospasm, gastralgia, bile reflux gastric ulcer, duodenal ulcer, irritable bowel syndrome, colonitis, gastrointestinal dysfunction, gastric kinetics disorder, indigestion, malabsorption and body weight loss, abdominal distension and diarrhea caused thereby.

13. A method of preventing or treating liver cell damage comprising administering to a human with liver cell damage a pufferfish type I collagen extract.

14. The method of claim 13 wherein the liver cell damage comprises damage selected from the group consisting of alcoholic liver damage, hepatic fibrosis, hepatic cirrhosis, alcohol-induced hepatic cirrhosis, alcohol-induced hepatic fibrosis, drug-induced liver damage, drug-induced hepatic fibrosis, and drug-induced hepatic cirrhosis.

15. A method of preventing or treating a collagen proliferative disease comprising administering to a human with a collagen proliferative disease a pufferfish type I collagen extract, wherein the proliferative disease is kidney fibrosis or myocardial fibrosis.

16. A method of preventing or treating an immune disease, comprising administering to a human with an immune disease a pufferfish type I collagen extract, wherein the immune disease is selected from the group consisting of immune dysfunction, leucopenia, rheumatoid arthritis, rheumatic arthritis, lupus erythematosus.

17. A method of preventing the development of or treating cancer, comprising administering to a cancer patient a pufferfish type I collagen extract, wherein the cancer is selected from the group consisting of malignant gastric tumors, gastric cancer, liver cancer, colon cancer, rectal cancer, solid malignant tumors.

18. A method preventing or treating the development of metastasis of a cancer, comprising administering to a cancer patient a pufferfish type I collagen extract, wherein the cancer is selected from the group consisting of malignant gastric tumors, gastric cancer, liver cancer, colon cancer, rectal cancer, solid malignant tumors.

19. A method of preventing or treating angiogenesis-associated diseases, comprising administering to a human with an angiogenesis-associated disease a pufferfish type I collagen extract.

20. The process according to claim 5, wherein the hydrolysis solution is neutralized, filtered and desalted, and then is purified by DEAE- and/or CM-ion exchange chromatography to remove other protein impurities, and the eluent of the ion exchange is desalted and dried to obtain a pufferfish type I collagen extract with a high purity.

21. The process according to claim 6, wherein the solution of redissolving the precipitate is neutralized, filtered and desalted, and then is purified by DEAE- and/or CM-ion exchange chromatography to remove other protein impurities, and the eluent of the ion exchange is desalted and dried to obtain a pufferfish type I collagen extract with a high purity.

22. The process according to claim 7, wherein the extract solution of claim 7 is neutralized, filtered and desalted, and then is purified by DEAE-and/or CM-ion exchange chromatography to remove other protein impurities, and the eluent of the ion exchange is desalted and dried to obtain a pufferfish type I collagen extract with a high purity.
